(19) United States
(12) Patent Application Publication
(10) Pub. No.: US 2025/0362294 A1
(43) Pub. Date: Nov. 27, 2025

(54) RAPID DETECTION TESTS AND METHODS OF FORMING THE SAME

(71) Applicant: XBIOLOGIX, INC., Corvallis, OR (US)

(72) Inventors: Eduardo Ceballos, Corvallis, OR (US); Qin Liu, Corvallis, OR (US)

(73) Assignee: XBIOLOGIX, INC., Corvallis, OR (US)

(21) Appl. No.: 18/865,410

(22) PCT Filed: May 16, 2023
(86) PCT No.: PCT/US2023/022381

§ 371 Date: Nov. 13, 2024

Related U.S. Application Data
(60) Provisional application No. 63/342,553, filed on May 16, 2022.

(51) Int. Cl. G01N 33/543

(52) U.S. Cl. CPC G01N 33/54388; G01N 33/54313; G01N 33/54353

(57) ABSTRACT

Examples are directed to rapid detection test (RDT) devices, apparatuses, and method of forming RDT device and/or apparatuses that include substrates functionalized with a coupling agent. An example RDT apparatus comprises a substrate at least partially coated with a coupling agent having functional groups, a test region disposed on a first portion of the substrate, the test region including a set of capture agents configured to bind to a target analyte in a biological sample, a control region disposed on a second portion of the substrate, the control region including a set of control agents, each of the control agents including an analyte protein, and a set of detection particles that exhibit a detectable label, each of the detection particles configured to bind to at least one of the target analyte or the analyte protein of the set of control agents.

### CROSS-REFERENCE TO RELATED APPLICATION

This application claims the benefit of U.S. Application No. 63/342,553, filed May 16, 2022, which is incorporated herein by reference in its entirety.

### BACKGROUND

Researchers are increasingly engaged in assay development and detection methods for specialized applications and instrumentation. A versatile platform for affinity assays or detection involves immobilizing antibodies or other proteins onto nitrocellulose surfaces. Various types of assays are used for different types of detection tests. A variety of biological samples may be tested using these various assays, including urine, saliva, sweat, serum, plasma, whole blood and other fluids or solids suspended in a fluid. Further, industries in which such assays may be employed include veterinary medicine, human medicine, quality control, product safety in food production, and environmental health and safety. In these areas of utilization, rapid tests are used to screen for animal diseases, pathogens, chemicals, toxins and water pollutants, among others.

### BRIEF DESCRIPTION OF THE DRAWINGS

FIG. 1 is a block diagram schematically illustrating an example rapid detection test (RDT) apparatus.

FIGS. 2A-2L are schematic illustrations of example regions of a RDT device and detection particles.

FIG. 3 is a block diagram schematically illustrating an example kit including an RDT device.

FIG. 4 is a block diagram schematically illustrating another example RDT apparatus.

FIGS. 5A-5C are schematic illustrations of example regions of another RDT device.

FIGS. 6-7 are block diagrams schematically illustrating different example methods of forming an RDT.

FIGS. 8-16 illustrate example results of forming at least portions of an RDT.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

In recent years there has been an increasing demand for point-of-care diagnostic or other detection tests to provide the rapid and simultaneous detection of a target analyte present in biological samples. It may be beneficial that such tests are easy to perform without the use of laboratory investigation, or individuals trained in chemical analysis. Moreover, transmission of pathogens such as influenza, severe acute respiratory syndrome coronavirus 2 (SARS-CoV-2) and others may persist and begin to circulate seasonally. For instance, with regards to COVID-19, (the disease caused by the SARS-COV-2 virus) sustained, widespread surveillance may be needed for several years to avoid resurgence. Many different rapid detection tests (RDT) may use an immunological assay that requires the lengthy and expensive screening of three different immunoglobulins antibodies by culturing proteins using cells. In many instances, the RDTs are manufactured by fabricating different substrates for the test line and the control line, a sample input and conjugated pad, among other portions, which are sometimes referred to a pads and which are assembled together onto an assembly with backing and packaged. Each substrate is uniquely processed, which requires time and increases the expense of the RDT. Further, the material type and overlapping dimensions need to be selected and controlled to make reproducible assays. The increase in time and expense may limit the availability and speed in RDTs being available for emerging pathogens.

Various examples are directed to an RDT apparatus, device, and/or kit, as well as method of forming the same, comprising a single substrate which is functionalized with a coupling agent with functional groups (e.g., reactive moieties) to allow for forming the different regions (e.g., test, control, and others) on the substrate. The different reagents for driving the test, including the coupling agent, may be deposited on the substrate, such as by digital printing and/or manually or other automated techniques, such as soaking or otherwise. The coupling agent may allow for the capture agents of a test region and control agents of a control region to bind to the substrate when deposited. The RDT may include detection particles which are each configured to bind to at least one of the target analyte and the control agents to provide an optical signal or other detectable signal to indicate the presence or not of the target analyte in the test region and indicate the test is operating normally via the control region. By using a single substrate, the RDT may be manufactured more quickly and using fewer steps, resulting in reduced costs as compared to RDT formed of multiple overlapping substrates.

Examples RDT devices and/or apparatuses described herein may include a (single) substrate which is functionalized with the coupling agent having functional groups and that includes a test region and a control region. The test region may include capture agents including a first ligand configured to bind to a target analyte in a biological sample. In some examples, the first ligand includes a protein which may bind to the functional groups of the coupling agent. In other examples, the capture agents further include a first linker that binds to the functional groups of the coupling agent and binds to the first ligand. The control region includes a set of control agents including an analyte protein. Similar to the first ligand, in some examples, the analyte protein may bind to the functional groups of the coupling agent. In other examples, the control agents further include a second linker that binds to the functional groups of the coupling agent and binds to the analyte protein. The RDTs may further include a set of detection particles, which may form part of the RDT device or may be separate therefrom and in a solution, as further described herein. The set of detection particles exhibit a detectable label, such as a visual color or fluorescence, and, in some examples, further include a bioorthogonal tethered protein or other label protein to enable the detection particle to act as a label, as further described herein.

As used herein, a test region refers to or includes a portion on the substrate where qualitatively assessing or quantitatively measuring the presence, amount, or functional activity of a target analyte may be performed. The capture agents refer to or include molecules or compounds which are bound (directly or indirectly) to the coupling agent and which are configured to bind to the target analyte. A control region refers to or includes a portion on the substrate where qualitative assessing of the functioning of the RDT may be performed. For example, the control region may be assessed to verify that the reagents function properly in the absence of the target analyte (e.g., did the test work or not). The control agents refer to or include molecules or compounds which are bound (directly or indirectly) to the coupling agent and which are configured to bind to the detection particles. Detection particles refer to or include particles which exhibit a detectable label (e.g., signal) and include a label ligand (e.g., protein) configured to bind to at least one of the target analyte and the control agents. In some examples, the particles include beads or nanoparticles which exhibit the detectable label. In some examples, the particles include the detectable label, such as a dye. In such examples, the detection particles include the detectable label and the label ligand, such as a label ligand that is tagged or labeled. In further examples, the particles include the label ligand itself which exhibits the detectable label, for example, being magnetic or otherwise exhibiting a charge. Accordingly, in some examples, the detection particles do not include a physical bead or nanoparticle and may be referred to as a detection element. As further described herein, the RDT devices and apparatuses may further include a sample input region which includes or refers to a portion of the substrate configured to receive a biological sample, and in some examples, may contain the set of detection particles. In some examples, the sample input region may include a sample sub-region to receive the biological sample and a conjugate sub-region that includes the set of detection particles.

In some examples, the RDT may be reduced in cost and improved in sensitivity by modifying protein ligands of interest to contain chemically reactive sites at specific locations on a protein. For example, by modifying the protein ligands, the proteins may be oriented on the surface of an RDT device so that the binding site is readily available for target analyte binding. Through the controlled modification of the test region chemistry, a ligand may be deposited in a uniform manner, reducing the potential for extraneous interference with target binding and overcoming issues with potential mass transport effects.

In some examples, a RDT apparatus or device of the present disclosure may include the use of noncanonical amino acids at least in a test region of the RDT, such as a noncanonical amino acid bearing a tetrazine moiety. A noncanonical amino acid bearing a tetrazine moiety may be selectively incorporated into a protein or a functional protein fragment to provide a tetrazine-modified protein at an amino acid site selected for modification. The tetrazine-modified protein may be tethered to a substrate surface via a linker, as described more thoroughly herein. More particularly, the tetrazine may be incorporated into a protein to enable covalent bonding of the protein to the substrate of the RDT in controlled concentration, orientation, length, and surface geometries. In some examples, the tetrazine may be incorporated to the protein to covalently bound to a particle to form a detectable particle. A substrate functionalized with a coupling agent may further enable covalent bonding of the tetrazine-modified protein to the substrate and multiple functions integrated into a single substrate. Furthermore, use of a tetrazine-modified protein may allow for deterministic loading of the reagents, which are dispense agnostic.

Nature uses a limited, conservative set of amino acids to synthesize proteins. This limited set includes 20 naturally-occurring amino acids, which are also referred to as canonical amino acids. Protein translation uses transfer ribonucleic acids (tRNAs), which are aminoacylated by aminoacyl-tRNA synthetase enzymes, to read triplet codons in messenger RNAs (mRNAs) via base pairing interactions between the mRNA codon and the anticodon of the RNA. The ribosome facilitates both the sequential decoding of triplet codons on mRNAs by cognate tRNAs, and the polymerization of the corresponding amino acids into a polypeptide. Unlike small organic molecule synthesis wherein almost any structural change may be made to influence functional properties of a compound, the synthesis of proteins is limited to changes encoded by the twenty natural amino acids. The genetic code of every known organism, from bacteria to human, encodes the same twenty common amino acids. These amino acids may be modified by posttranslational modification of proteins, e.g., glycosylation, phosphorylation or oxidation, or in rarer instances, by the enzymatic modification of aminoacylated suppressor tRNAs, e.g., in the case of selenocysteine. Nonetheless, polypeptides, which are synthesized from only these 20 simple building blocks, carry out all of the complex processes of life. By expanding the genetic code to include additional amino acids with novel biological, chemical or physical properties, the properties of proteins, e.g., the size, acidity, nucleophilicity, hydrogen-bonding, hydrophobic properties and reactivity, may be modified as compared to a protein composed of only amino acids from the 20 common amino acids, e.g., as in a naturally occurring protein.

Noncanonical amino acids have been developed that undergo rapid and selective reactions in cells through inverse electron demand Diels-Alder reactions between strained alkenes or alkynes and tetrazines. One technique in the peptide modification is based on the introduction of genetically encoded noncanonical amino acids into proteins via bioorthogonal tRNA/tRNA-synthetase pairs. In combination with site-specific incorporation of noncanonical amino acid into proteins via genetic code expansion, reactions have emerged as valuable tools for labeling and manipulating proteins in living systems. Reactions have found application for imaging of cell-surface and intracellular proteins, for labeling and identifying proteomes in E. coli, mammalian cells, and multicellular organisms as well as for selectively inhibiting a specific target protein within living cells.

Approaches have been developed to expand the genetic code, enabling the co-translational and site-specific incorporation of diverse noncanonical amino acids into proteins synthesized in cells. These noncanonical amino acids are not naturally-occurring amino acids, and therefore expand the genetic code beyond the limited set of 20 naturally-occurring amino acids. As used herein, a noncanonical amino acid refers to or includes an amino acid that is not naturally-occurring, and therefore not among the list of 20 naturally-occurring amino acids. A non-limiting example of a noncanonical amino acid of the present disclosure includes an amino acid that has been genetically encoded to include a tetrazine moiety at a predetermined amino acid site. The phrase “genetically encoded to include a tetrazine moiety at a predetermined amino acid site” refers to or includes a process by which a noncanonical amino acid bearing a tetrazine moiety is selectively incorporated into a protein or a functional protein fragment to provide a tetrazine-modified protein or a tetrazine-modified functional protein fragment at an amino acid site selected for modification. The genetic encoding method may be used to incorporate a noncanonical amino acid bearing a tetrazine moiety during in-cellulo protein synthesis, and/or in a cell-free protein synthesis environment. The genetic encoding method may be used to incorporate a noncanonical amino acid bearing a tetrazine moiety at any site (e.g., amino acid position) in the protein or a functional protein fragment. By virtue of the position of the tetrazine moiety in the tetrazine-modified protein or the tetrazine-modified functional protein fragment, and because of the selective reactivity of the tetrazine moiety with a trans-cyclooctene-modified surface, the orientation of the protein or functional protein fragment on the surface may be controlled. The method allows for control of the presentation of the protein or functional protein fragment on the surface.

As used herein, a protein refers to or includes a molecule comprising chains of amino acids, and which may fold into a three dimensional structure. Proteins, such as the tetrazine-modified protein, analyte protein, target analyte, and/or label protein, are not limited to the full protein and may include functional protein fragments. Accordingly, as used throughout, a tetrazine-modified protein may include and/or be referred to as a tetrazine-modified functional protein fragment. Similarly, an analyte protein may include or be referred to as an analyte functional protein fragment and a label protein may include or be referred to as a label functional protein fragment.

As used herein, “orthogonal” or “biorthogonal” refers to or includes a molecule (e.g., an orthogonal tRNA (O-tRNA) and/or an orthogonal aminoacyl tRNA synthetase (O-RS)) that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal may refer to an inability or reduced efficiency, e.g., less than 20% efficient, less than 10% efficient, less than 5% efficient, or less than 1% efficient, of an orthogonal tRNA to function with an endogenous tRNA synthetase compared to an endogenous tRNA to function with the endogenous tRNA synthetase, or of an orthogonal aminoacyl-tRNA synthetase to function with an endogenous tRNA compared to an endogenous tRNA synthetase to function with the endogenous tRNA. The orthogonal molecule lacks a functional endogenous complementary molecule in the cell. For example, an orthogonal tRNA in a cell is aminoacylated by any endogenous RS of the cell with reduced or even zero efficiency, when compared to aminoacylation of an endogenous tRNA by the endogenous RS. In another example, an orthogonal RS aminoacylates any endogenous tRNA in a cell of interest with reduced or even zero efficiency, as compared to aminoacylation of the endogenous tRNA by an endogenous RS. A second orthogonal molecule may be introduced into the cell that functions with the first orthogonal molecule. For example, an orthogonal tRNA/RS pair includes introduced complementary components that function together in the cell with an efficiency (e.g., 50% efficiency, 60% efficiency, 70% efficiency, 75% efficiency, 80% efficiency, 90% efficiency, 95% efficiency, or 99% or more efficiency) to that of a corresponding tRNA/RS endogenous pair.

In some examples, a substrate of a RDT device or apparatus may include a particular concentration of tethered (e.g., linked) tetrazine-modified protein that promotes a high rate of analyte binding without inhibition from neighboring ligands. As described further herein, the concentration of tetrazine-modified protein on the substrate may be selected as a function of the analyte to be detected, and therefore, is specific for the test to be performed. In some examples, the binding site of each of the tetrazine-modified proteins may be oriented in a same direction and in a direction specific for the analyte to be detected (e.g., the target analyte). By orienting the binding domains of the tetrazine-modified proteins in a particular direction, the binding affinity of the analyte increases, allowing for a more sensitive assay. Yet further, a length of the ligand may be specifically selected to promote analyte binding and limit cross-reactivity with the substrate surface or other surface chemistries.

In various examples, a substrate may be formed, which includes a bioorthogonal tethered protein in at least one portion of the substrate (e.g., test region and/or control region). The bioorthogonal tethered protein may be formed on a substrate by attaching a tetrazine-modified protein to a linker. As used herein, a bioorthogonal tethered protein refers to or includes a tetrazine-modified protein that has been attached to a linker. Also as used herein, a tetrazine-modified protein refers to or includes a protein or functional protein fragment that contains a tetrazine. The bioorthogonal tethered protein may include a ligand configured to bind to a target analyte. A concentration, a length, and an orientation of the bioorthogonal tethered protein may be configurable on the substrate. As used herein, a ligand refers to or includes a molecule that binds to another molecule. A target analyte refers to or includes a molecule that binds to a ligand, and which the RDT may be designed to detect. A linker refers to or includes a molecule that binds to the tetrazine-modified protein or another analyte protein and binds to coupling agent on the substrate.

The tetrazine-modified protein may be prepared by genetic encoding using a noncanonical amino acid bearing a tetrazine moiety. In some examples, the length, concentration, and orientation of the bioorthogonal tethered protein are selected based on the target analyte to be detected.

Although various examples described above include the use of a tetrazine-modified protein as reagents in different regions of the RDT device and/or apparatus, examples are not so limited and may include other types of capture agents and control agents.

In some examples, the sensitivity of the RDT may be improved or further improved via the use of detection particles. The detection particles may be deposited onto the substrate, in a sample input region, or may be located in solution in which the biological sample is input to and then placed on the RDT. The detection particles may each include a particle that exhibits a detectable label (e.g., colorant, fluorescent, or other detectable signal) and includes at least a label protein, such as a tetrazine-modified protein. The label protein may be configured to bind to one or both of the target analyte and the control agents. In some examples, multiple different detection particles may be used. For example, different sized particles bound to same type of tetrazine-modified protein may be used to capture additional light or other signals.

As may be appreciated, many diagnostic approaches begin testing after a patient is symptomatic. As an illustration, an infectious disease may have a 3-day latent period in which a patient is infected but asymptomatic. At this point, the patient may have approximately 100 copies of a viral protein in a sample. At day 5, the patient begins exhibiting symptoms, and on day 9 the patient obtains a test. The patient may not receive the results from their testing until around day 14 (e.g., 14 days after they became infected), at which point the patient may have as many as 10\hat{ }6 copies of the viral protein in a sample. Throughout the entire 14 day period, the patient has been infected and capable of transmitting the infection to persons nearby. As such, testing for a pathogen after a patient begins to display symptoms does not prevent the spread of the infection. Detecting early enough to stop the spread requires testing and diagnosis before symptoms appear, such as when the limit of detection for the pathogen is around 100 virus copies per sample. Accordingly, a need exists for a portable assay device, that is specific for detecting a particular analyte, sensitive enough to detect small volumes of the analyte, and scalable for mass-production and use in a point-of-care setting. Although the above-describes a viral pathogen and diagnostics, examples are not so limited and may be directed to other pathogens or analytes and/or for detection purposes other than diagnostics. Other example pathogens include bacteria, fungi, protozoa, worms. Example analytes, which may or may not be a pathogen, include radioactive material or components, enzymes, toxins, pollutants, food allergens, among others.

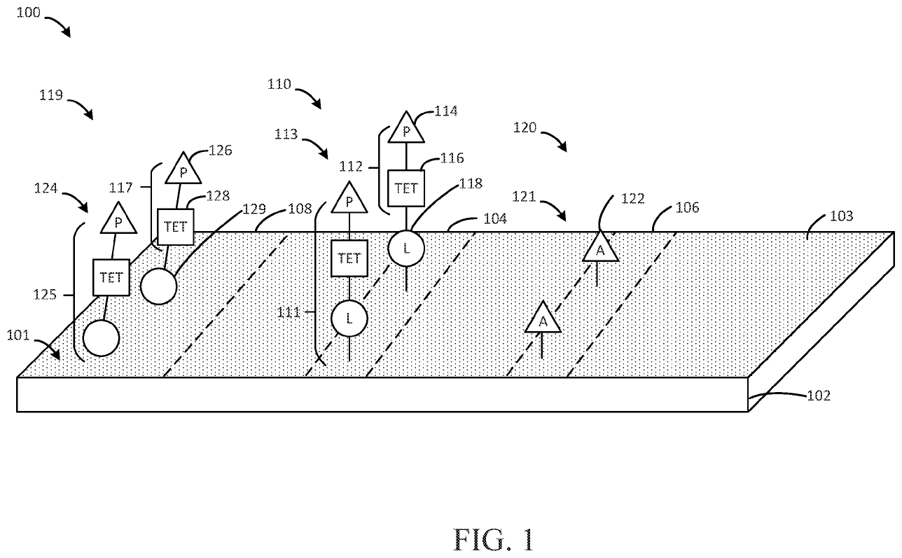

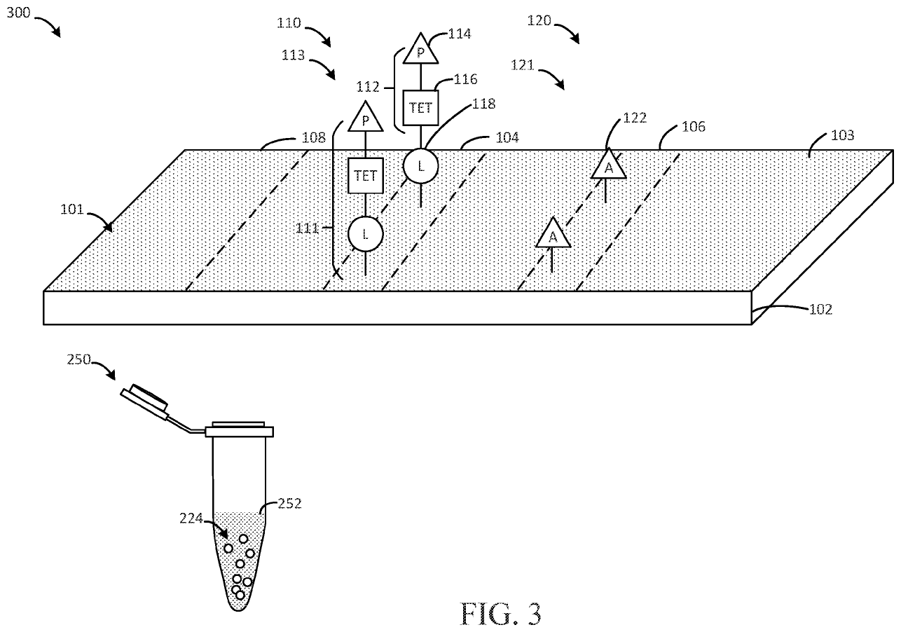

Turning now to the figures, FIG. 1 is a block diagram schematically illustrating an example rapid detection test (RDT) apparatus. The apparatus 100 may include or be an RDT device 101. In some examples, the apparatus 100 may further include other components, such as detection particles in solution as illustrated by FIG. 3.

In some examples, the RDT device 101 includes a substrate 102, a test region 110, and a control region 120. As used herein, a substrate refers to or includes a solid or porous substance that receives the deposited layers of molecules. The substrate 102 may be at least partially coated with a coupling agent 103. In some examples, the entire substrate is coated or covered with the coupling agent 103.

In some examples, the substrate 102 is formed of glass microfibers (GMF), a polymer (e.g., plastic), or a metal. In some examples, the substrate 102 is formed of or includes a membrane, such as a mesh membrane. In further examples, the substrate 102 may be formed of one or more of glass, GMF, a polymer, polypropylene, paper, metal, metal fibers, carbon nanotube fibers (CNTF), non-woven material, plasma treated material, and silicon.

The coupling agent 103 may be deposited to at least a portion of the substrate 102. For example, the coupling agent 103 may be deposited to at least a first portion 104 and a second portion 106 of the substrate 102. In some examples, the coupling agent 103 is further deposited to a third portion 108 or to all of the top surface of the substrate 102 or the entire substrate 102. As used herein, a coupling agent refers to or includes a molecule or compound that may be used to provide a chemical bond between two materials, such as two dissimilar materials like glass and an organic molecule or compound (e.g., substrate and tetrazine-modified protein). For example, the coupling agent 103 may be modified to include the functional group, e.g., moieties that are added to the compound. The coupling agent 103 has functional groups to which other molecules or compounds, e.g., the capture agents 113 and control agents 121, may bind to. In some examples, the coupling agent 103 comprises a compound or molecule functionalized (bound to a functional group selected from) with at least one of an epoxide functional group, a carboxylate functional group, an anhydride functional group, and an amine functional group, among other functional groups.

In some examples, the coupling agent 103 may be silane. For example, the coupling agent 103 comprises a silane coupling agent which may be functionalized with at least one of an epoxide functional group, a carboxylate functional group, an anhydride functional group, and an amine functional group. However examples are not so limited and may include other functional groups.

The coupling agent 103 may allow for other molecules or compounds to bind to the substrate 102 by binding to the functional groups of the coupling agent 103, which may otherwise not be capable of binding to the substrate 102 or bind at below a threshold rate. For example, the coupling agent 103 may create more surface area for a linker or other molecule or compound to bind to. In some examples, the modification or functionalization process, e.g., silanization process, may include using trimethoxysilane with NaOH pre-treatment, as further described herein. In some examples, for GMF or microfiber membranes substrates, an epoxide, an amine, a carboxylic acid, and/or an anhydride functional silane may be used. In addition, some portions of the substrate 102 may be selectively treated with other coupling agents to afford surfaces with less tendency for non-specific binding.

In some examples, the test region 110 is disposed or formed on a first portion 104 of the substrate 102. The test region 110 may include a set of capture agents 113 configured to bind to a target analyte in a biological sample. In some examples, each of the capture agents of the set of capture agents 113 include first bioorthogonal tethered proteins 111 including a first tetrazine-modified protein 112 and a first linker 118. The first tetrazine-modified protein 112 may include a first ligand 114 configured to bind to the target analyte. More particularly, in some examples and as shown, the first tetrazine-modified protein 112 includes a tetrazine 116 bound to the first ligand 114.

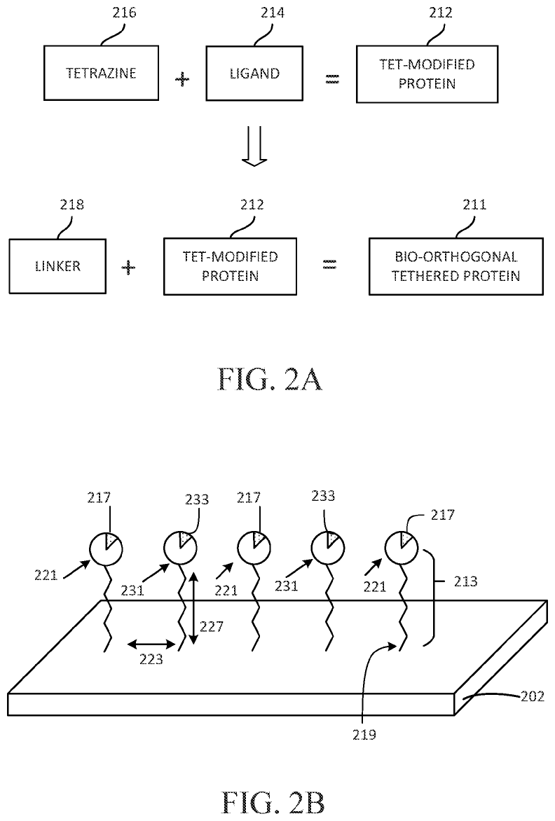

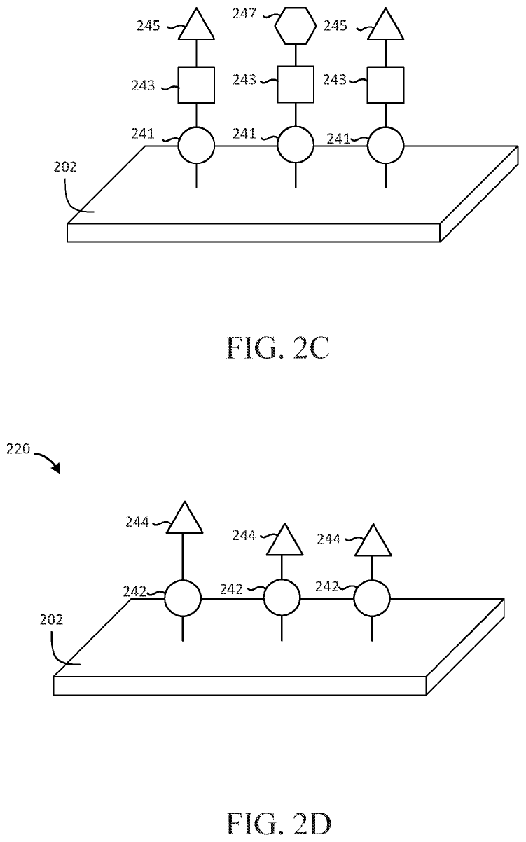

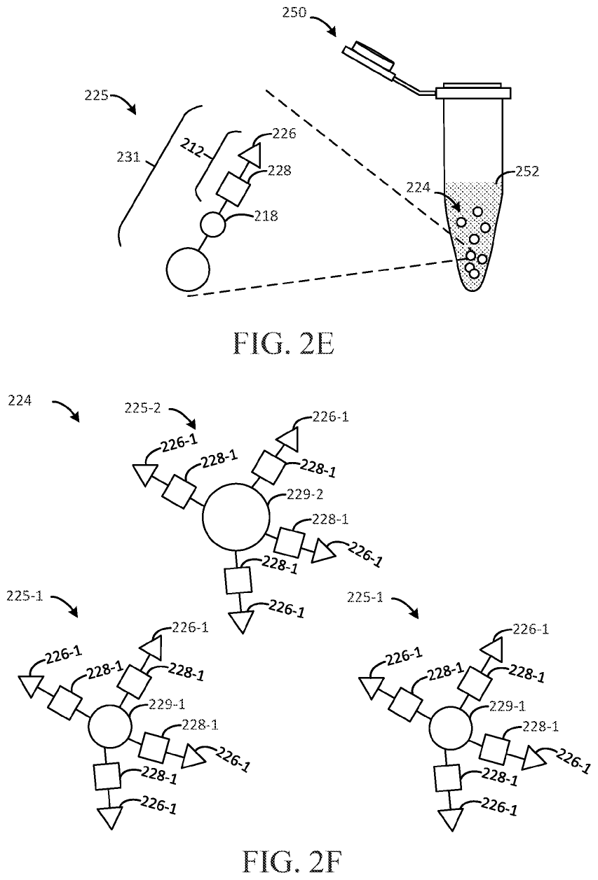

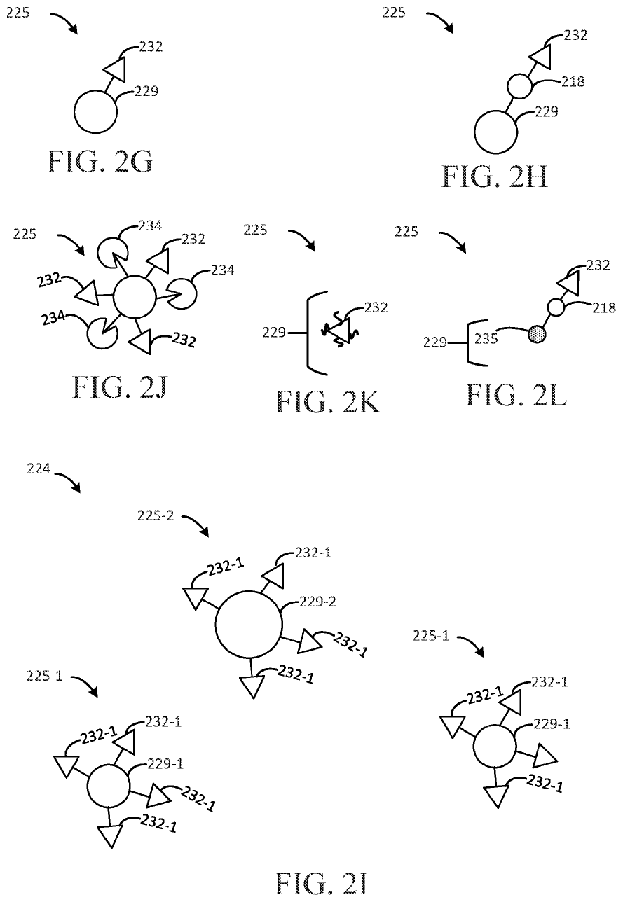

The set of capture agents 113 may include a first linker covalently bound to the tetrazine-modified protein 112 and to functional groups of or associated with the coupling agent 103 disposed in the test region 110. In various examples, the first linker 118 (and optional second linker and/or third linker, as further illustrated by FIGS. 2D-2H) includes a trans-cyclooctene (TCO) including a TCO derivative, e.g., sTCO, with functional groups (e.g., moieties), for example, a TCO with an amine moiety, a TCO with a carboxylic acid moiety, a norbornene anhydride, a norbornene with an amine moiety, and/or a norbornene with a carboxylic acid moiety, among other molecules.

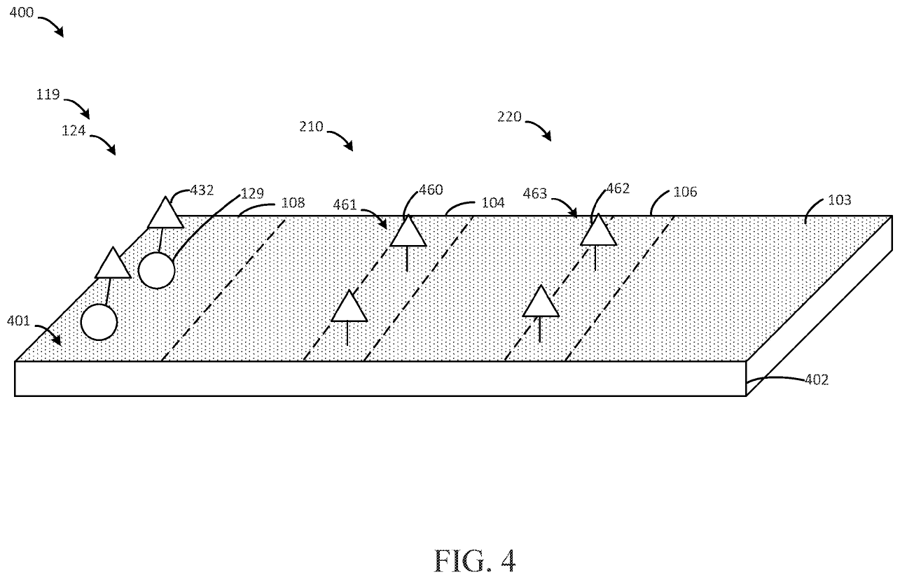

Further details of bioorthogonal tethered proteins is provided below, at least in connection with FIGS. 2A-2C. As used herein, the phrase “tethered” refers to or includes attaching a protein to another protein or surface by a number of bond modalities. Although FIG. 1 illustrates use of bioorthogonal tethered proteins as the capture agents, examples are not so limited. In some example, other types of proteins may be used, which bind directly or indirectly to the coupling agent 103 in the test region 110, as further illustrated by FIG. 4.

In some examples and as noted above, the first tetrazine-modified protein 112 covalently binds to the first linker 118, which may maintain avidity of the protein (e.g., first ligand 114), sometimes referred to as the “orthogonality”. In some examples, multimers of the first tetrazine-modified protein 112 may be prepared with one or more tetrazine moieties at a pre-selected location on the protein to control the length and orientation of the protein when immobilized on a substrate 102 (or the surface of a detection particle as further described herein). In some examples, specificity of the covalent bonding and the speed of reaction also allows for the control of loading at predetermined concentration and controlled (partial) loading on substrates (e.g., substrate 102 or detection particles 124). The first ligand 114 (and analyte protein 122) may be a variety of different types of proteins and protein fragments, and are not limited to immunoglobulin G (IgG) or IgM. For example, a functional fragment of a protein may be used. As another example, nanobodies may be used. Use of proteins that are different from IgG may allow for faster manufacturing. In some examples, the first ligand 114 and analyte protein 122 may be made in cultures other than mammalian cells, such as E. coli cells, which may be faster (e.g., 3 times faster than mammalian cells) and less expensive (e.g., 1000 times less expensive than mammalian cells).

In some examples, a control region 120 is disposed or formed on a second portion 106 of the substrate 102. The control region 120 may include a set of control agents 121, each of the control agents including an analyte protein 122. In some examples, the analyte protein 122 may be bound directly to the coupling agent 103 present in the second portion 106 of the substrate 102. For example, the analyte protein 122 may include a reactive moiety that may react with the functional groups of or associated with the coupling agent 103 on the substrate 102. In other examples, as shown by FIG. 2D, the analyte protein 122 may be bound indirectly to the coupling agent 103 via a second linker bound to the coupling agent 103. The analyte protein 122 refers to or includes a protein configured to bind to a detection particle (e.g., via a label protein) and is bound to the substrate 102 in the control region 120, as described below. As used herein, the analyte protein 122 may be interchangeably referred to as “a control protein” and is not the target analyte. In some examples, the analyte protein 122 includes a second bioorthogonal tethered protein. In other examples, the analyte protein 122 may not include a bioorthogonal tethered protein as the control may not require optimization for low detection limit. Similarly, protein fragments or proteins other than (and smaller than) IgG may be used as the analyte protein 122.

The linker(s) may or may not be present in the test region 110 and/or the control region 120 (and/or on the set of detection particles 124) depending on the functional group(s) on the protein (e.g., first ligand 114 and analyte protein 122) that is immobilized to the substrate 102 (and/or the particle). In the case of tetrazine-modified protein, a linker may be used. A linker may help differentiate what site the protein is immobilized to the substrate 102 with a corresponding reactive moiety contained in the protein. Controlling the site where a protein is immobilized to the substrate 102 can be useful for maintaining avidity and orientation of the protein. In some examples, a linker may not be used if the protein contains a reactive moiety that reacts with the functional groups of the coupling agent 103 on the substrate 102 faster than those inherent to the amino acids in the protein. In some examples, if one is not striving for the utmost detection limit, where the site(s) on the protein immobilized to the substrate 102 is not as important, a linker may not be used.

The apparatus 100 may further include a set of detection particles 124 that exhibit a detectable label. As previously described, detection particles refer to or include particles which exhibit a detectable label (e.g., signal) and include a label protein configured to bind to at least one of the target analyte and a respective control agent of the set of control agents 121. The detectable label includes or refers to a property or signal which may be detected, such as a visual color, optical signal (e.g., fluorescence), electrical or magnetic property, among other labels which may be detected. In some examples, the detectable label may include a dye and/or each of the set of detection particles 124 may include a label protein bound to or otherwise labeled with the dye. Non-limiting examples of a dye includes a fluorescent dye or other types of colorant.

In various examples, each detection particle 125 of the set of detection particles 124 includes a second tetrazine-modified protein 117 including a second ligand 126 configured to bind to at least one of the target analyte and the analyte protein 122 of the set of control agents 121. Similar to the first tetrazine-modified protein 112, the second tetrazine-modified protein 117 may include a tetrazine 128 bound (e.g., via a carbon link) to the second ligand 126. More particularly, in some examples, the detection particles 125 include a particle 129 (e.g., bead) with the second tetrazine-modified protein 117 bound thereto. Although not illustrated by FIG. 1, in some examples, each detection particle 125 of the set of detection particles 124 may further include a (third) linker bound between the second tetrazine-modified protein 117 and the particle 129. The first linkers associated with the set of capture agents 113, and optional second linkers associated with the set of control agents 121 and/or third linkers associated with the set of detection particles 124 may include a same type of linker or different types of linkers, and combinations thereof. In other examples, as further illustrated by at least FIG. 2L, the detection particles 125 each include a particle comprising a dye bound to a label protein (via a linker), such as the second tetrazine-modified protein 117 bound to a fluorescent dye via a linker.

In some examples, the set of detection particles 124 are disposed on a sample input region 119 configured to receive the biological sample, wherein the test region 110 and control region 120 are downstream from the sample input region 119 of the substrate 102. The set of detection particles 124 may be deposited via digital printing, as further described herein. In such examples, the set of detection particles 124 form part of the RDT device 101. As such, various examples are directed to an RDT device 101 that include the substrate 102, the test region 110, the control region 120, and the sample input region 119 including the set of detection particles 124.

In some examples, the sample input region 119 may include two sub-regions juxtaposed together to form the sample input region 119. The two sub-regions may include a sample sub-region to receive the biological sample and a conjugate sub-region that includes the set of detection particles 124. The two sub-regions of the sample input region 119 may be formed of the same material (e.g., the substrate 102 and the coupling agent 103) with the conjugate sub-region being further treated with the set of detection particles 124. The conjugate sub-region may be downstream of the sample sub-region. In some examples, the sample sub-region may be overlapping, e.g., all or portions thereof, with the conjugate sub-region or may be on top of the conjugate sub-region.

In other examples, the set of detection particles 124 may be initially separate from the RDT device 101, and may form part of a kit that includes the RDT device 101 as further illustrated by FIG. 4.

In some examples, the set of detection particles 124 are disposed on a sample input region 119. The sample input region 119 is formed on a third portion 108 of the substrate 102 which is upstream from the test region 110 and the control region 120.

In some examples, the set of detection particles 124 may be deposited onto the substrate 102, such as in the third portion 108 of the substrate 102. The set of detection particles 124 may be deposited via digital printing or analog dispensing. In some examples, the set of detection particles 124 may further include a sugar moiety to maintain an avidity of the second tetrazine-modified protein 117, as further described herein.

In some examples, each of the set of detection particles 124 is a gold nanoparticle (AuNP) functionalized with at least one of the second tetrazine-modified protein 117. In other examples, each of the set of detection particles 124 is a latex nanoparticle functionalized with at least one of the second tetrazine-modified protein 117. In some examples, the latex nanoparticles include a colored, fluorescent, magnetic, or paramagnetic latex particle. In other examples, each of the set of detection particles 124 includes the label protein and a dye, and may not include a physical nanoparticle or bead.

The various lines in FIG. 1 (and in other figures illustrated herein) shown between respective molecules or compounds, such as the line between the first ligand 114 and tetrazine 116, are schematic illustration of binding between the respective molecules or compounds. The molecules or components may be bound together biological and/or chemically. In some examples, the set of detection particles 124 may not be bound to the substrate 102, and may be otherwise be connected to the substrate 102 such as being disposed or placed thereon. In some examples, the set of detection particles 124 may bind temporarily to the substrate 102, such as by binding via a sugar matrix or other dissolvable binding agent. In some examples, based on the depositing of the set of detection particles 124, the set of detection particles 124 may be formed in a stack or include multiple layers of detection particles. In such examples, a first portion of the set of detection particles 124 may be connected to the substrate 102 and the remaining portions may be layered on top of the first portion.

In various examples, use of the second tetrazine-modified protein 117 bound to (or forming part of) the particles as the detection particles may allow for controlled loading which enables more particles to be connected to the test region 110, which may be referred to as controlled partial loading. For example, the particles may be bound to a volume of the second tetrazine-modified protein 117 that is lower than a maximum volume that may be loaded on the particles (or loaded onto the substrate 102), which may increase the lower detection limit for the RDT device 101. Said differently, the controlled (partial) loading may result in less than 100 percent loading of the second tetrazine-modified protein 117 (or other label protein) on the particle 129 (or onto the substrate 102). The controlled (partial) loading may reduce the likelihood of multiple target analytes binding to the same detection particle, and conversely, increase the likelihood that multiple target analytes bind to different detection particles (thereby increasing the detection signal). In response, a greater number of detection particles may be bound to a limited number of available target analytes in the biological sample, which are subsequently captured on the test region 110 to enhance the signal for (low) concentrations of the target analyte.

In some examples, each detection particle 125 of the set of detection particles 124 is attached to the second tetrazine-modified protein 117 having the same type of second ligand 126 (e.g., targets or binds to the same antigen). In some examples, the set of detection particles 124 include particles 129 of different sizes.

In some examples, blocking agents may be deposited on the test region 110, the control region 120, the sample input region 119, and/or on at least some of the detection particles 125 of the set of detection particles 124. As used herein, a blocking agent refers to or includes a molecule or compound that blocks (e.g., prevents, mitigates, or slows down) non-specific binding in the test region 110 or in the control region 120, or that aids in the release of the detection particles 124 when deposited in the sample input region 119. Non-limiting example blocking agents include casein, bovine serum albumin (BSA), 5X Detector™, polyethylene glycol, non-ionic surfactants, among other blocking agents. In some examples, the blocking agents may include compounds that react with the coupling agent 103 on the substrate 102 and modify the surface or react with the linker(s) (e.g., the first linker 111 and, optional, second linker) to be less prone to non-specific binding by itself or when used in combination with another (e.g., traditional) blocking agent, for the whole substrate 102 or selected regions, e.g., the conjugate sub-region of the sample input region 119. For example, the blocking agent(s) may be used across the entire substrate 102, in particular regions, on at least some or all of the detection particles, and/or not at all. In some examples, the blocking agent(s) may react with the functional group of the substrate 102, the linker (e.g., first linker 111), and/or is non-reactive.

In some examples, the RDT apparatus 100 may include components for providing flow control, which may be referred to as “a flow control agent”. As used herein, a flow control agent includes and/or refers to material incorporated to modulate (e.g., increase and/or decrease) the flow rate of fluids, such as the sample including the analyte and/or other components. The flow rate of the analyte may be modulated to enhance the detection signal from the detectable label of the set of detection particles 124 while minimizing overall test time. Some examples include temporary (physical) barriers placed in the path of the analyte flow, e.g., sugar, or putting a barrier in combination with modulating viscosity of the biological fluid including the sample, e.g., polyethylene glycol (PEG), methylcellulose, or modulate the path of the flow by way of modifying the hydrophobicity of certain regions on the substrate, such as applying polycaprolactone (PCL), or a combination thereof.

The apparatus 100 and/or RDT device 101 illustrated by FIG. 1 and various figures herein may be used to implement different types of RDTs. In some examples, the RDTs include a flow test, such as a lateral flow test or a dipstick test. Examples are not limited to flow test and may include other types of RDTs.

Various examples include the use of tetrazine-modified proteins in at least one portion of the RDT device 101, which may allow for deterministic loading. This allows for use of smaller proteins and for the capture agent and label protein (e.g., tetrazine-modified protein used in the detection particles) to be developed in bacterial cell lines and thus shortens the development time and lowers the cost of the development and expression of the proteins. Tetrazine-modified protein may be covalently bonded to a functionalized substrate and largely maintain its avidity. This makes the development of the proteins for a RDT more deterministic. This also allows for optimization of the detectable signal through controlled deposition of a label protein on the particles to form detection particles (e.g., controlled partial loading) and capture agents on the test region to maximize the number of particles associated with each analyte protein and caption at the test region.

In some examples, the functionalized substrates provide the functionality for a tetrazine-modified protein to be covalently bonded to the substrate. It also allows a single substrate to be used and facilitates digital printing of multiple reagents, which reduce the time and cost for manufacturing the substrate and the RDT device and/or apparatuses. Digital deposition enables less reagent deposition of expensive reagents. In some examples, printing of a reagent may include double pass printing, such as with the set of detection particles 124.

In some examples, a silane coupling agent may be used to functionalize GMF substrates and yield functionalized GMF substrates with functional groups (e.g., reactive moieties) such as, epoxide, carboxylic acid, anhydride, amine, etc. For membranes of other materials, other functionalization processes may be used. For example, grafting of glycidyl methacrylate to nitrocellulose membrane through electron beam irradiation may be used. These functional groups or reactive moieties on the substrate can be used further to modify the surface of the substrate in selected areas to provide a linker to a protein that contains unique moieties for orthogonal covalent bonding, to react/interact with a blocking agent for the remaining area to prevent non-specific binding, and to react with another compound to affect the flow rate of fluid. The reactive moieties on the linker, the blocking agent, and/or the other compound may be those that react or interact with the functional groups of the coupling agent on the substrate.

Accordingly, in some examples, the linker used may be dependent on the functional groups on the functionalized substrate. For example, a TCO with an amino moiety used as a linker, a substrate that contains an epoxide, a carboxylic acid (or its derivative using 1-Ethyl-3-(3-(dimethylamino) propyl) carbodiimide (EDC)/N-hydroxysulfosuccinimide (sNHS)), or an anhydride may be used (e.g., may react with). Conversely, if the reactive moiety on the functionalized substrate is an amine, a carboxylic or an anhydride or an epoxide moiety may be used in the TCO. The selection of the linker may be dependent on the non-canonical amino acid incorporated into the tetrazine-modified protein. For example, a TCO or a norbornene may be used as linkers for a tetrazine-modified protein. Conversely, if TCO is incorporated into the protein as a part of the non-canonical amino acid, tetrazine may be the linker to link the protein to the substrate. Another example of the linker-non-canonical amino acid pairing is azide and alkyne, where one may serve as a linker to the other that is incorporated into a protein in the form of the non-canonical amino acid.

Various examples are directed to use of blocking agents and/or physical barrier to impact the flow rate. Using the same substrate for different functions in a RDT may make it difficult to the control of flow rate. Examples of a reactive compound that modifies the flow characteristics of a silane functionalized GMF, as further described herein.

Various examples are directed to a single substrate that functions similarly to a traditional RDT, but without the use of multiple substrate which are assembled together. Each RDT, e.g., reagents forming the RDT, may be dispensed directly onto the single substrate with the appropriate surface modifications. An alternative to the RDT being dispensed onto the substrate, components may be combined with the sample at the time of testing and are wicked up the substrate for a test result, such as with a dipstick flow test and/or other tests.

FIGS. 2A-2L are schematic illustrations of example regions of an RDT device and detection particles. The various regions and/or components illustrated by FIGS. 2A-2L may be implemented in the apparatus 100 and/or device 101 of FIG. 1, and/or the kit 300 and/or apparatus 400 and/or device 401 of FIG. 3 or 4.

FIG. 2A is a schematically illustration of an example of forming a bioorthogonal tethered protein 211. The bioorthogonal tethered protein 211 may be used to form the capture agents and/or control agents, as described by FIG. 1. As further described herein, in some examples, the detection particles may include a bioorthogonal tethered protein.

In some examples, the bioorthogonal tethered protein 211 may be formed on a substrate by attaching a tetrazine-modified protein 212 to a linker 218. The bioorthogonal tethered protein 211 may include a ligand 214 configured to bind to a target analyte and a tetrazine 216. As discussed herein, a concentration, a length, and an orientation of the bioorthogonal tethered protein may be configurable on the substrate. In various examples, the substrate may be configurable in the types, and amounts, of analytes that may be detected (e.g., bound) to the surface. For instance, the substrate may be configured to detect and/or bind a single analyte in a sample (such as the SARS-COV2 virus), and/or may be configured to detect a plurality of analytes in the sample (such as the SARS-COV 2 virus, influenza A, and influenza B).

Protein translation uses transfer ribonucleic acids (tRNAs), which are aminoacylated by aminoacyl-tRNA synthetase enzymes, to read triplet codons in messenger RNAs (mRNAs) via base pairing interactions between the mRNA codon and the anticodon of the tRNA. In the example illustrated in FIG. 2A, a noncanonical amino acid such as a tetrazine 216 or tetrazine moiety may be site-specifically incorporated with a ligand 214 to form a tetrazine-modified protein 212 by attaching the tetrazine 216 or tetrazine moiety to a selector codon (e.g., STOP codon) of a gene. The resultant combination of the tetrazine 216 or tetrazine moiety with the ligand 214 is a tetrazine-modified protein 212.

The ligand 214 may include a protein or other receptor molecule that may bind to the target analyte. As used herein, a ligand includes a molecule that binds to a target analyte or other target, such as an analyte protein. An analyte includes the molecule that is being detected and/or measured, such as protein of a virus, compound, and/or other pathogens. For more general information on proteins attached to tetrazine, and specific information on example tetrazine structures, reference is made to US Patent Publication 2019/0077776, published on Mar. 14, 2019, and entitled “Reagents and methods for bioorthogonal labeling of biomolecules in living cells”, and PCT Publication WO/2015/054658, published on Apr. 16, 2015, and entitled “Modified amino acids comprising tetrazine functional groups, methods of preparation, and methods of their use”, which are herein incorporated by reference in their entirety for their teachings.

The tetrazine-modified protein 212 may be prepared by genetic encoding using a non-canonical amino acid bearing a tetrazine moiety. For instance, referring to FIG. 2A, the tetrazine-modified protein 212 may be genetically encoded to include a ligand 214. Using an orthogonal aminoacyl-tRNA synthetase and an orthogonal tRNA, the noncanonical amino acid (in this case, a tetrazine or tetrazine moiety) a tetrazine-modified protein or tetrazine-modified functional protein fragment may be prepared that includes both the tetrazine 216 (or tetrazine moiety) and the ligand 214. In various examples, the ligand 214 includes a fragment or portion of a protein. For instance, a fragment of protein A may comprise the ligand 214, and the fragment of protein A may be genetically encoded to include the tetrazine 216 or tetrazine moiety to generate the tetrazine-modified protein 212.

FIG. 2B is block diagram schematically illustrating an example region of substrate, in accordance with the present disclosure. The region of the substrate may include a test region and/or a control region of the RDT device. As described with regards to FIG. 1, various RDT apparatuses and/or devices include a bioorthogonal tethered protein 213 which is deposited on a substrate 202 by attaching a tetrazine-modified protein to a linker 219. The tetrazine-modified protein may be formed by contacting a tetrazine molecule with a ligand (e.g., 221, 231). The combination of the linker and tetrazine-modified protein may provide for ordered deposition of the ligand. For example, the ordered deposition may result in each ligand being positioned with the analyte binding site in a particular orientation. In some examples, the analyte binding site of each ligand may be facing or standing up. Through the controlled orientation, each binding site of the bound protein may be oriented in the same way, which may provide for optimization of the binding affinity (e.g., Kd) and which may prevent or mitigate non-specific binding and/or provide a 100-1000 fold improvement in detection indicator signal. TCO, as used herein, may include TCO and/or strained TCO (sTCO).

In some examples, the linker 219 is deposited on the substrate 202, and the tetrazine-modified protein is then deposited on the substrate 202. In other examples, the bioorthogonal tethered proteins 213 are formed and then deposited.

As illustrated in FIG. 2B, a plurality of bioorthogonal tethered proteins 213 may be tethered to the substrate 202. Each bioorthogonal tethered protein 213 includes a ligand 221 capable of binding an analyte 217, and a linker 219. In some examples, the length, concentration, and orientation of the bioorthogonal tethered protein 213 are selected based on the target analyte to be detected. For instance, the plurality of bioorthogonal tethered proteins 213 may be formed on the substrate 202 in such a manner that the orientation of a binding domain of each of the ligands 221 is facing in a same direction (as illustrated). Similarly, the plurality of bioorthogonal tethered proteins 213 may be formed on the substrate 202 in a manner such that the concentration 223 of the bioorthogonal tethered proteins 213 on the substrate 202 allow for each of the bioorthogonal tethered proteins 213 to bind to a target analyte 217. Yet further, the plurality of bioorthogonal tethered proteins 213 may be formed on the substrate 202 in a manner such that the length 227 of the bioorthogonal tethered proteins 213 on the substrate 202 allow for each of the bioorthogonal tethered proteins 213 to bind to a target analyte 217. Orientation of the ligands may be useful for a variety of tests.

In some examples, the bioorthogonal tethered protein 213 are selectively formed on the substrate 202. As further described herein, forming the bioorthogonal tethered protein 213 includes depositing a coupling agent to at least a portion of the substrate 202 and contacting the linker 219 with the coupling agent. As a specific example, a silane coupling agent may be deposited on the substrate 202 and the substrate 202 may be treated with TCO, resulting in the TCO binding to the silane coupling agent.

As illustrated in FIG. 2B, a plurality of different ligands 221, 231 may be tethered to the substrate 202 in a test region, which allows for a multiplexed test.

For instance, the substrate 202 may include a first tetrazine-modified protein that includes a first ligand 221, and a second tetrazine-modified protein that includes a second ligand 231. The first tetrazine-modified protein or including the first ligand 221 may bind to a first analyte 217. The second tetrazine-modified protein including the second ligand 231 may bind to a second analyte 233. By combining multiple tetrazine-modified proteins on a same substrate, an RDT device of the present disclosure may increase the dynamic range of detection and/or detection of all isotypes on a single assay device. In addition, RDT devices of the present disclosure may include proteins of different affinities for a given analyte, which also provides for a wider range of binding activity on the RDT device. As such, in some examples, forming the bioorthogonal tethered protein includes contacting a first tetrazine-modified protein with the linker, and contacting a second tetrazine-modified protein with the linker. In some examples the first analyte and the second analyte may be different, such that different analytes may be detected on a same assay device.

FIG. 2C is a block diagram schematically illustrating an example substrate with a tetrazine-poly(ethylene glycol) (tet-PEG) polymer, where the tet-PEG polymer is used as a blocking agent. Similar to FIG. 2B, the region of the substrate 202 may include a test region and/or control region. As is further similar to the example illustrated in FIG. 2B, the substrate 202 includes a plurality of bioorthogonal tethered proteins. The tet-PEG may prevent non-specific binding to particular aspects of the substrate 202, in effect reducing signal noise. For example, the region of the substrate 202 illustrated in FIG. 2C includes three tethered molecules. Each of the tethered molecules may include a linker 241 tethered to the substrate 202, and a tetrazine or tetrazine fragment 243 tethered to the linker 241. In some examples, as illustrated by FIG. 2C, tet-PEG and tetrazine-modified protein are in parallel, both tethered to the substrate 202 via the linker 241. A poly(ethylene glycol) (PEG) molecule 245 may be tethered to a tetrazine or tetrazine fragment 243. The third bioorthogonal tethered protein illustrated in FIG. 2C includes a ligand 247 tethered to a tetrazine or tetrazine fragment 243. The PEG molecule 245 may optionally be attached to remaining linkers 241 that are not attached to the tetrazine-modified protein, e.g., 243 and 247, and which may mitigate or prevent non-specific binding of the target analyte. The PEG polymer may provide a wider dynamic range and allow for keeping the sample undiluted, such that quantitative results may be obtained.

Example are not limited to using PEG as a blocking agent. For instance, in some examples, an ethanolamine may be used to block amine reactive sites. Other example blocking agents may include compounds that include amino moieties that may be used to block amine reactive sites introduced in the substrate functionalization process, such as 1-butylamine. Conversely, if the reactive moiety/functional group introduced in the functionalization process is carboxylic acid or anhydride reaction, the blocking agents may contain carboxylic acid or anhydride. If the linker is less prone to non-specific binding, the blocking agent application step for the linker may not be included (e.g., the substrate or a portion thereof may not include a blocking agent or an additional blocking agent).

As used herein, designations of “first” and “second” are used to refer to one element and another of the same element, of the same type or of a different type, without reference to temporal order. As such, a first portion of the linker may be tethered to a ligand whereas a second portion of the linker may be tethered to PEG or another blocking agent, without reference to a temporal order of deposition. In some examples, the order of deposition of ligand and PEG may be specified.

For instance, and as a specific non-limiting example, the agents may be loaded on the RDT device by first treating the surface of the substrate 202 with NaOH, followed by trimethoxysilane and which results in trimethoxysilane bound on the surface. The surface may then treated with TCO-NH2, resulting in TCO bound to the silane. For example, once the trimethoxysilane is reacted, trimethoxy groups may are no longer be present. The surface may then treated with the tetrazine-modified protein and the tet-PEG polymer (if relevant), resulting in the tetrazine-modified protein being tethered to a portion of the volume of TCO and tet-PEG polymer being bound to the remaining portion of the volume of TCO (if relevant).

Accordingly, in some examples, forming the bioorthogonal tethered protein includes depositing a volume of the linker 241 to the substrate 202 in a test region of the RDT device, and optionally in a control region of the RDT device. The method may further include attaching the tetrazine-modified protein, e.g., 243 and 247 illustrated in FIG. 2C, to at least a first portion of the linker 241. The method may optionally include attaching a tet-PEG polymer to a second portion of the linker 241.

In some examples, the bioorthogonal tethered protein includes the tetrazine-modified protein or tetrazine-modified functional protein fragment in a configured orientation. As used herein, “bioorthogonal” may include or refer to the amino acid tether embedded in the protein structure having no (or minimal) effect on the folding or activity of the ligand. In some examples, the amino acid tether may be at any site of the ligand, and as such, the orientation of the protein may be controlled. For example, the ligand protein may be oriented so it binds optimally with its binding partner (e.g., by putting the tether on the far side of the protein, relative to the binding domain); or the opposite, and the tether may be placed close to the binding domain (so that the binding domain faces the surface, as far as possible from receptor domain of the binding partner). As the orientation of the ligands follow from the site-specific attachment of the tetrazine and the bioorthogonal properties, each of the ligands that are immobilized may be oriented in the same way on the surface of the substrate 202, resulting in a uniform biomolecular mono-layer of biologic agents. Also, as used herein, the term “bioorthogonal tethered” or “bioorthogonally tethered” with regards to the protein refers to or includes a protein that contains a tetrazine moiety, or other type of moiety, which is capable of being tethered to a surface or has been tethered to a surface bioorthogonally.

As described above, in some examples, the control agents of the control region may include a tetrazine-modified protein, and may include at least some of the same features and attributes as described by FIGS. 2B-2C. For example, in some examples, each of the set of control agents includes a second bioorthogonal tethered protein and the analyte protein includes a third tetrazine-modified protein (which forms part of the second bioorthogonal tethered protein). As previously described, the control region may be used to verify that the reagents function properly in the absence of the analyte. In the case of detecting IgG, on the test region, various chemistries may be used to immobilize anti-IgG on the membrane and tested with the protein binding assay. Similarly, on the control region, various chemistries may be used to immobilize an IgG used to bind to an anti-IgG forming part of the detection particles. The anti-IgG loading and hydrophilicity (which determines the incubation time) of the membrane may be optimized to obtain the maximum color intensity.

However, examples are not so limited and either or both of the control region and the test region may not include tetrazine-modified proteins. For example, the control agents may include other types of ligands that bind to the substrate (directly or indirectly through a linker) that includes other types of proteins. Similarly, the capture agents may include other types of ligands that bind to the substrate (directly or indirectly through a linker) and are specific for a particular antigen.

FIG. 2D is a block diagram schematically illustrating an example control region 220. As previously described, in some examples, each of the control agents of the control region 220 may include an analyte protein 244 and a second linker 242. The second linker 242 may be bound to the analyte protein 244 and to the second portion of the substrate 202 (e.g., to the coupling agent). As previously described, a linker may be used when the analyte protein 244 is unable to bind to the functional group of the coupling agent and/or binds at a rate below a threshold. In some examples, the analyte protein 244 may not readily bind in a proper orientation while retaining a threshold (e.g., sufficient to bind to its target) avidity due to the size of the protein. As the analyte protein 244 decreases in size, there may be less opportunities to randomly orient the analyte protein 244 in a way the analyte protein 244 is able to capture or bind to a detection particle 225.

FIG. 2E illustrates an example of a set of detection particles 224. In some examples, the apparatus 100 of FIG. 1 further includes a sample container 250 that includes (e.g., stores) a solution 252 with the set of detection particles 224, the sample container 250 is configured to receive the biological sample and to provide the biological sample and the set of detection particles 224 to a sample input region of the substrate.

Some examples are directed to a detection particle, such as the particular detection particle 225 illustrated by FIG. 2E. In some examples, the detection particle 225 includes at least one tetrazine-modified protein 212 bound to a surface of the particle 229 that exhibits a detectable label (e.g., colorant or other detectable signal). As described above, the tetrazine-modified protein 212 includes a tetrazine 228 bound to a ligand 226 configured to bind to at least one target. In some examples, the ligand 226 is configured to bind to both of the target analyte and the control agent in the control region. In other examples, the ligand 226 is configured to bind to one of the target analyte and the control agent in the control region. As described above, the particle 229 may be an AuNP, a latex, or other nanoparticle that is functionalized with the at least one tetrazine-modified protein 212. The detection particle 225 may contain more tetrazine-modified protein 212 than illustrated.

In some examples, the detection particle 225 and/or each detection particle of the set 224 includes a (third) linker, as further illustrated herein.

FIG. 2F illustrates another example set of detection particles 224. In some examples, the set of detection particles 224 may include the same size particles or different sized particles.

The particles sizes and/or ligands may be adjusted to increase sensitivity of the RDT. In some examples, the sensitivity is increased by at least one of: (i) the isometrics and controlled orientation/geometry due to use of the tetrazine-modified proteins or other ligands; (ii) controlled (partial) loading of the tetrazine-modified proteins or other label proteins on the particles; and/or (iii) use of the detection particles.

For example, multiple different sized detection particles may be used to increase the sensitivity. The limited of detection (LOD) may be dependent on a number of detection particles which are captured by the test region. Different sized particles bound to same type of label protein, such as a tetrazine-modified protein, may be used to capture additional light and increase the sensitivity.

In some examples, the flow of the set of detection particles 224 may be adjusted or controlled. For example, respective ones of the set of detection particles 224 may include one or more sugars, which may reduce the flow rate of the detection particles with the one or more sugars. Reducing the flow rate of the set of detection particles 224 may be used to increase the binding efficiency or rate associated with forming complexes between respective ones of the set of detection particles 224 with respective ones of the target protein and respective ones of the set of capture agents on the test region.

As an example illustrated by FIG. 2F, the set of detection particles 224 may include a first type of detection particle 225-1 and a second type of detection particle 225-2. The first type of detection particle 225-1 may include a particle 229-1 bound to a first tetrazine-modified protein including a tetrazine 228-1 and a first ligand 226-1 configured to bind to at least one of the target analyte and the control agents. The second type of detection particle 225-2 may include a particle 229-2 bound to a first tetrazine-modified protein including a tetrazine 228-1 and the first ligand 226-1 configured to bind to at least one of the target analyte and the control agents, wherein the particle 229-2 is larger than the particle 229-1. Although not illustrated by FIG. 2F, the tetrazine-modified proteins may be indirectly linked to the particles 229-1, 229-2 via linkers, such as the linker 218 illustrated by FIG. 2E. The kit may include more or fewer detection particles 225-1, 225-2 and/or more or fewer first tetrazine-modified proteins bound to each particle 229-1, 229-2 than illustrated.

Various examples are directed to a kit comprising a solution (e.g., 252 of FIG. 2E) and a set of detection particles that exhibit a detectable label (e.g., 224 of FIGS. 2E-2F). Each of the detection particles include a tetrazine-modified protein, wherein tetrazine-modified protein includes a tetrazine bound to a ligand configured to bind to at least one target. As described above, each detection particle of the set of detection particles is a gold or latex nanoparticle functionalized with at least one of the tetrazine-modified protein. In some examples, each detection particle of the set of detection particles is attached to the tetrazine-modified protein having the same type of ligand. In some examples, respective ones of the set of detection particles includes particles of different sizes. In some examples, each of the detection particles further include a second linker bound to the second tetrazine-modified protein and the particle.

Examples are not limited to detection particles as illustrated by FIGS. 2E-2F and may include variations, such as detection particles with use of linkers. FIG. 2G illustrates an example of a detection particle 225 that includes at least one label protein 232 bound to a surface of the particle 229 that exhibits a detectable label. The label protein 232 may include or is a ligand configured to bind to at least one target (e.g., the target analyte and/or the control agents). The label protein 232 may be bound directly to the particle 229 or indirectly via a linker. Accordingly, although not illustrated, a linker may be bound to the label protein 232 and to the particle 229.

FIG. 2H illustrates an example a detection particle 225 that includes at least one label protein 232 bound indirectly to a surface of the particle 229 that exhibits a detectable label via a linker 218. As shown, the linker 218 is bound to the label protein 232 and to the particle 229.

Various examples are directed to a kit comprising a solution and a set of detection particles that exhibit a detectable label, similar to that described above. FIG. 2I illustrates another example set of detection particles 224. In some examples, the set of detection particles 224 may include the same size particles or different sized particles 229-1, 229-2, with the particles 229-1, 229-2 including attached first label proteins 232-1, similar to that described above in connection with FIG. 2F. The kit may include more or fewer detection particles 225-1, 225-2 and/or more or fewer first label proteins 232-1 bound to particles 229-1, 229-2 than illustrated. Similarly, the detection particle may contain more label proteins or tetrazine-modified proteins bound thereto than illustrated by any of FIGS. 2E-2J.

In some examples, at least some of the detection particles may include a blocking agent. FIG. 2J illustrates an example detection particle 225 that includes at least one label protein 232 bound (directly or indirectly via a linker (not shown)) to a surface of the particle 229 that exhibits a detectable label and at least one blocking agent 234. Although not shown, the label proteins 232 may include tetrazine-modified proteins, in some examples, and/or may be bound to the particle 229 via a linker, thereby forming bioorthogonal tethered proteins. Further, although the label protein 232 and the blocking agent 234 are illustrated in equal volume (e.g., three each), examples are not so limited and may include more or fewer of the label protein 232 and the blocking agent 234 than illustrated.

In some examples, at least some of the detection particles may include a label protein and detectable label, without a nanoparticle or bead. That is, the detection particles may each include a particle formed of or including the label protein and detectable label. FIG. 2K illustrates an example detection particle 225 that includes a particle 229 formed of or including the label protein 232 and the detectable label, such as a dye and as illustrated by the lines on the label protein 232. In some such examples, the label protein 232 may be connected directly to a substrate of the RDT device (e.g., a sample input region), connected via a linker or other component (e.g., dye) the detection particle 225, and/or may be in solution. FIG. 2L illustrates an example detection particle 225 that includes a particle 229 formed of or including the detectable label 235, with the detection particle including a label protein 232, the detectable label 235, and a linker 218 bound between the label protein 232 and the detectable label 235, as further described herein. In some such examples, the detectable label 235 may include a dye, such as a fluorescent dye. In some examples, the detection particle 225 may include a 1:1 ratio of detectable label to label protein. In some examples, the ratio associated with the detectable label 235 (such as with nanoparticles) may be different than a 1:1 ratio of detectable label to label protein, such as 1:2 or more.

FIG. 3 is a block diagram schematically illustrating an example kit including an RDT device. As shown, the kit 300 may include an RDT device 101, which may include at least some of substantially the same features and attributes as the RDT device 101 of FIG. 1, as shown by the common numbering and with the common features and attributes not being repeated for ease of reference. The kit 300 may further include a sample container 250 including a solution 252 and a set of detection particles 224, which may include at least some of substantially the same features and attributes as the sample container 250 of FIG. 2E, as shown by the common numbering and with the common features and attributes not being repeated for ease of reference.

FIG. 4 is a block diagram schematically illustrating another example RDT apparatus. As previously described, examples are not limited to RDT apparatuses and devices comprising test regions and/or control regions with bioorthogonal tethered proteins. Various features and attributes of the apparatus 400 may include at least substantially the same features and attributes of the apparatus 100 of FIG. 1, as shown by the common numbering and with the details of the common features and attributes not being repeated.

As shown by FIG. 4, the apparatus 400 comprises a GMF substrate 402 at least partially coated with a silane coupling agent 103 having functional groups, such as any of the previously described silane coupling agents. In some examples, the fibers of the GMF substrate 402 have a diameter of less than about 10 um, although examples are not so limited.

The test region 210 may be disposed on a first portion 104 of the substrate 402. The test region 210 includes a set of capture agents 461 configured to bind to a target analyte in a biological sample. In some examples, each of the capture agents include a first ligand 460 configured to bind to the target analyte. As further described below, the first ligands 460 may be directly or indirectly (via a first linker) bound to the silane coupling agent 103.

The control region 220 may be disposed on a second portion 106 of the substrate 402. The control region 220 includes a set of control agents 463. In some examples, each of the control agents including an analyte protein 462. As further described below, the analyte protein 462 may be directly or indirectly (via a second linker) bound to the silane coupling agent 103.

The apparatus 400 may further include a set of detection particles 124 that exhibit a detectable label. As previously described, each of the detection particles may include a label protein 432, wherein the label protein 432 includes a second ligand configured to bind to at least one of the target analyte and the analyte protein 462 of the set of control agents. The set of detection particles 124 of the apparatus 400 of FIG. 4 may include any of the variations as described above in connection with FIG. 1, FIGS. 2E-2L, and FIG. 3. As further described below, the label protein 432 may be directly or indirectly (via a first linker) bound to the silane coupling agent 103.

As with the apparatus of FIG. 1, in some examples, the set of detection particles 124 are disposed on a sample input region 119 configured to receive the biological sample. In such examples, the set of detection particles 124 form part of the RDT device 401 which includes the substrate 402, the test region 210, the control region 220, and the sample input region 119.

In other examples, the set of detection particles 124 are separate from the RDT device 401. For example, the apparatus 400 may further include a sample container that includes a solution with the set of detection particles 124, as illustrated by FIG. 3. In such examples, the apparatus 400 may include or form part of a kit that includes the RDT device 401 and the sample container.

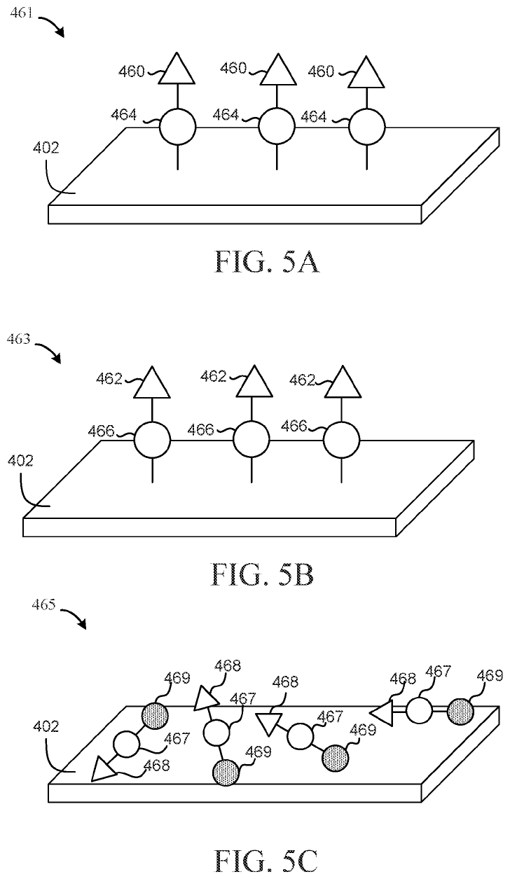

FIGS. 5A-5C are schematic illustrations of example regions of another RDT device, such as the RDT device 401 of FIG. 4. As previously described, a linker may or may not be present in the capture agents and control agents of the RDT device of the apparatus of FIG. 4, depending on what functional group on the protein is immobilized to the substrate 402.

In some examples, the first ligand 460 and/or the analyte protein 462 are capable of binding to the functional group of the silane coupling agent 103 and are bound directly to the silane couple agent 103. In other examples, as illustrated by FIG. 5A, each of the capture agents 461 further include a first linker 464 bound to the silane coupling agent 103 in the first portion of the substrate 402, wherein the first ligand 460 is bound to the first linker 464. In some examples, as shown by FIG. 5B, each of the control agents 463 further include a second linker 466 bound to the silane coupling agent 103 in the second portion of the substrate 402, wherein the analyte protein 462 is bound to the second linker 466. In some examples, each of the detection particles, such as the set of detection particles 124 in FIG. 4, may further include a third linker bound to the particles and bound to the label protein. In some examples, as illustrated by FIG. 5C, each of the detection particles 465 may include a label protein 468, a detectable label 469, and a third linker 467 between the label protein 468 and the detectable label 469, where at least a portion of the detection particles 465 are connected (temporarily, such as interface in a surface-to-surface manner) to the silane coupling agent 103 in the first portion (or other portion) of the substrate 402. As previously described, in some examples, the detection particles 465 may be deposited to form layers of detection particles, with a first portion being connected to the silane coupling agent 103 (or directly to the substrate without a silane coupling agent 103) and other portions being connected to the first portion (or more layers). As previously described, the first, second, and third linkers may include the same type or different types of linkers.

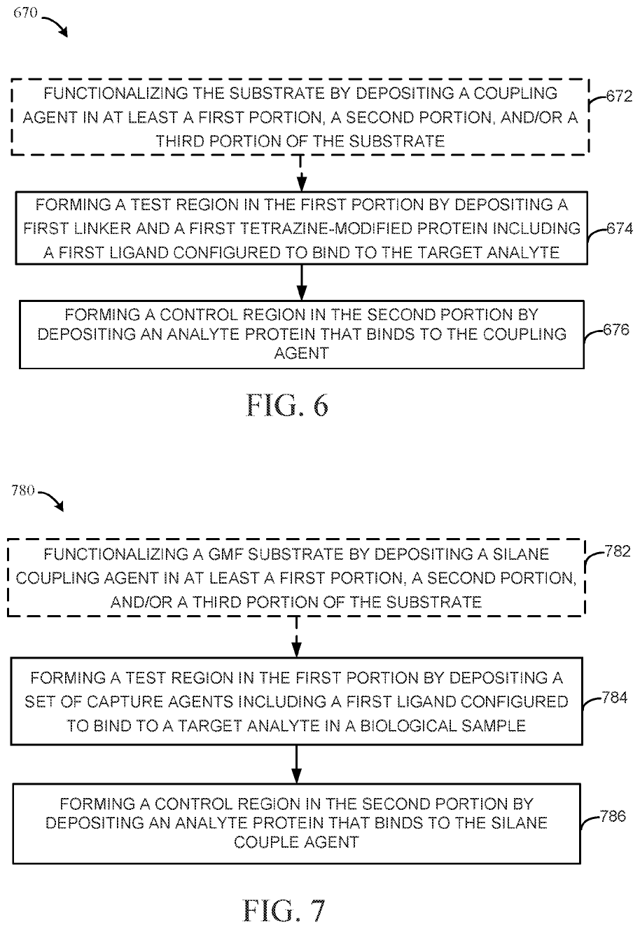

FIGS. 6-7 are block diagrams schematically illustrating different example methods of forming an RDT. The reagents (e.g., coupling agent, capture agents, control agents, and, optionally detection particles and linkers) may be deposited on the substrate digitally via printing or analog. Digital printing may dial in the amount of typically expensive reagents and reduce waste. In addition, digital printing provides additional controls over analog dispensing. The placement of the drops may be optimized in the X-Y plane. The penetration of the reagent into the substrate may be optimized through the selection of drop volume, number of drops placed at a given site (number of passes) for a given total volume. These optimizations may directly or indirectly lead to enhanced signals. A single functionalized substrate makes printing down various reagents more convenient and economical.

FIG. 6 shows an example method 670 of forming a substrate, such as any of the substrates of the apparatuses, devices, and/or kits previously described herein. As shown at 672, the method 670 includes functionalizing the substrate by depositing a coupling agent in at least a first portion, a second portion, and/or, a third portion of the substrate. In some examples, the coupling agent is applied to the entire substrate. As shown at 674, the method 670 includes forming a test region in the first portion by depositing a set of capture agents each including: (i) a first linker to the first portion of the substrate, wherein the first linker binds to the coupling agent present in the first portion of the substrate; and (ii) a first tetrazine-modified protein including a first ligand configured to bind to the target analyte, wherein the first tetrazine-modified protein binds to the first linker to form a first bioorthogonal tethered protein. In some examples, the first linker is first deposited, followed by the first tetrazine-modified protein. In other examples, the first linker and the first tetrazine-modified protein are deposited together, where the first bioorthogonal tethered protein is formed prior to deposition. As shown at 676, the method 670 further includes forming a control region in the second portion by depositing a set of control agents each including an analyte protein that binds (e.g., directly or indirectly through a linker) to the coupling agent.

In some examples, the analyte proteins directly bind to the coupling agent and, in other examples, the set of control agents each further include a second linker. For example, the analyte protein binds to the coupling agent via a second linker and forming the control region further comprises depositing the second linker to the second portion of the substrate, wherein the second linker binds to the coupling agent present in the second portion of the substrate and the analyte protein binds to the second linker.

In some examples, the method 670 further comprises forming a sample input region in the third portion of the substrate by: (i) attaching second tetrazine-modified proteins to particles to form a set of detection particles, the second tetrazine-modified proteins each including a second ligand configured to bind to at least one of the target analyte and the analyte protein of the set of control agents; and (ii) depositing the set of detection particles to the third portion of the substrate to form the sample input region, such as forming the conjugate sub-region of the sample input region.

In some examples, the method 670 further comprises attaching second tetrazine-modified proteins to particles to form a set of detection particles, the second tetrazine-modified proteins each including a second ligand configured to bind to at least one of the target analyte and the analyte protein of the set of control agents, and dispersing the set of detection particles in solution.

In some examples, the second tetrazine-modified proteins directly bind to the particles and, in other examples, the detection particles each further include a third linker. For example, the set of detection particles include the second tetrazine-modified proteins configured to bind to the surface of the particles via a third linker and forming the sample input region further comprises depositing the linker to the surface of the particles, wherein the third linker binds to the particles and to the second tetrazine-modified proteins.

In some examples, the method 670 further includes depositing at least one blocking agent to at least one region of the RDT device. However examples are not so limited. For example, forming the test region, at 674 in FIG. 6, may further include depositing a first blocking agent to the test region after depositing the first linker and before depositing the first tetrazine-modified protein. In some examples, forming the control region at 676 further includes depositing a second blocking agent (which may be the same type of agent as applied in the test region or a different type) to the control region after depositing the second linker and before depositing the analyte protein. In some examples, forming the sample input region may further include depositing a third blocking agent to the conjugate sub-region after depositing the coupling agent and prior to depositing the set of detection particles. In some examples, the method 670 may include depositing a fourth blocking agent to at least a portion or all of the set of detection particles, such as after depositing a (second or third) linker and before depositing the second tetrazine-modified protein to the particles. In some examples, the method 670 may include depositing the first blocking agent to the test region and the second blocking agent to the control region, the first blocking agent to the test region and the third blocking agent to the sample input region, the first blocking agent to the test region and the fourth blocking agent to the particles, and/or depositing each of the first blocking agent, the second blocking agent, the third blocking agent, and the fourth blocking agent, and various combinations thereof.

In various examples, at least one of the depositions of the method 670 includes digitally dispensing the reagents using inkjet printing (e.g., piezo, thermal, or continuous inkjet (CIJ)) or other inkjet printing technologies. Examples are not so limited and may include other dispensing techniques.

FIG. 7 shows an example method 780 of forming a substrate, such as any of the substrates of the apparatuses, devices, and/or kits previously described herein. As shown at 782, the method 780 includes functionalizing a GMF substrate by depositing a silane coupling agent in at least a first portion, a second portion, and/or a third portion of the substrate. In some examples, the silane coupling agent is applied to the entire GMF substrate. As shown at 784, the method 780 includes forming a test region in the first portion by depositing a set of capture agents each including a first ligand configured to bind to a target analyte in a biological sample, wherein the set of capture agents bind to the silane coupling agent. As shown at 786, the method 780 further includes forming a control region in the second portion by depositing a set of control agents each including an analyte protein that binds to the silane coupling agent, wherein the analyte protein forms a capture agent.

In some examples, the first ligands directly bind to the silane coupling agent and, in other examples, the set of capture agents each further include a (first) linker. For example, the first ligand may bind to the silane coupling agent via a linker and forming the test region further comprises depositing the linker to the first portion of the substrate, wherein the linker binds to the silane coupling agent present in the first portion of the substrate and the first ligand binds to the linker.

In some examples, the analyte proteins directly bind to the silane coupling agent and, in other examples, the set of control agents each further include a (second) linker. For example, the analyte protein may bind to the silane coupling agent via a linker and forming the control region further comprises depositing the linker to the second portion of the substrate, wherein the linker binds to the silane coupling agent present in the second portion of the substrate and the analyte protein binds to the linker.

In some examples, the method 780 further comprises forming a sample input region in the third portion of the substrate by: (i) attaching label proteins to particles to form a set of detection particles, the label proteins each including a second ligand configured to bind to at least one of the target analyte and the analyte protein of the set of control agents; and (ii) depositing the set of detection particles to the third portion of the substrate to form the sample input region, such as to the conjugate sub-region.

In some examples, the method 780 further comprises attaching label proteins to particles to form a set of detection particles, the label proteins each including a second ligand configured to bind to at least one of the target analyte and the analyte protein of the set of control agents, and dispersing the set of detection particles in solution.

In some examples, the label proteins directly bind to the particles and, in other examples, the detection particles each further include a (third) linker. For example, the label protein may bind to the surface of the particles via a linker and forming the sample input region further comprises attaching the linker to the surface of the particles and attaching the label proteins to the linker.

Similarly to the method 670, the method 780 may further include depositing at least one blocking agent to at least one region of the RDT device. However examples are not so limited. For example, forming the test region, at 784 in FIG. 7, may further include depositing a first blocking agent to the test region after depositing the first linker and before depositing the first ligands. In some examples, forming the control region at 786 further includes depositing a second blocking agent (which may be the same type of agent as applied in the test region or a different type) to the control region after depositing the second linker and before depositing the analyte protein. In some examples, forming the sample input region may further include depositing a third blocking agent to the conjugate sub-region after depositing the silane coupling agent and prior to depositing the set of detection particles. In some examples, the method 780 may include depositing a fourth blocking agent to at least a portion or all of the set of detection particles, such as after depositing a (second or third) linker and before depositing the second label protein to the particles. In some examples, the method 780 may include depositing the first blocking agent to the test region and the second blocking agent to the control region, the first blocking agent to the test region and the third blocking agent to the sample input region, the first blocking agent to the test region and the fourth blocking agent to the particles, and/or depositing each of the first blocking agent, the second blocking agent, and the third blocking agent, and the fourth blocking agent, and various combinations thereof.

In various examples, at least one of the depositions of the method 780 includes digitally dispensing the reagents using inkjet printing (e.g., piezo, thermal, or CIJ) or other inkjet printing technologies. Examples are not so limited and may include other dispensing techniques.

While the example methods 670, 780 of FIGS. 6-7 are described as including functionalizing the substrate and/or GMF substrate by depositing a silane coupling agent, examples are not so limited. In various examples, the substrate may be pre-functionalized, such that there is not depositing of the silane coupling agent in the methods 670, 780. As such, the steps 672, 782 of the methods 670, 780 are optional, as illustrated by the dashed lines in FIGS. 6-7. For example, a supplier of the substrate may deposit the silane coupling agent and provide the substrate including the silane coupling agent on (e.g., deposited on) at least a portion thereof.

In various examples, a RDT device may be formed, which includes the substrate described herein. For instance, a RDT device (also referred to herein as an “assay device”) may start with a liquid sample (or its extract) containing a target analyte. The liquid sample may move without the assistance of external forces (capillary action) through various zones on which molecules may interact with the RDT device. Non-limiting examples of an RDT device may include a test strip, a microfluidic device with microfluidic channels, and other assay devices. Example assays include lateral flow assay (LFA), chemiluminescent immunoassays, among other plate assays or other types of assay tests, such as isotopic immunoassay, fluoroimmunoassay, radioimmunoassay, microbiologic assays, quantal or graded bioassays, and others. The sample may be applied at one end of the RDT device, and the sample may migrate through the various zones in the RDT device, and recognition of the analyte results in a response on the test region, while a response on a control region indicates the proper liquid flow through the RDT device. The sample may also be introduced to the test region without going through various zones by being directed immediately to a test region. The read-out, which may indicate a qualitative or quantitative assessment of the analyte, may be assessed by eye or using a dedicated reader. In order to test multiple analytes simultaneously under the same conditions, additional test regions of ligands specific to different analytes may be immobilized in an array format. On the other hand, multiple test regions loaded with the same ligand may be used for semi-quantitative assays.

The phrase “genetically encoded to include a tetrazine moiety at a predetermined amino acid site” refers to the process described herein by which a non-canonical amino acid bearing a tetrazine moiety is selectively incorporated into a protein or a functional protein fragment to provide a tetrazine-modified protein or a tetrazine-modified functional protein fragment at an amino acid site selected for modification. The genetic encoding method described herein may be used to incorporate a non-canonical amino acid bearing a tetrazine moiety at any site (e.g., amino acid position) in the protein or a functional protein fragment. By virtue of the position of the tetrazine moiety in the tetrazine-modified protein or the tetrazine-modified functional protein fragment, and because of the selective reactivity of the tetrazine moiety with the linker-modified surface, the orientation of the protein or functional protein fragment on the surface is controlled. The methods allows for control of the presentation of the protein or functional protein fragment on the surface.

In some examples, forming a RDT device as described herein may include patterning the substrate to include a plurality of regions including the test region and a plurality of channels connected to the test region using a hydrophobic or hydrophilic material, and depositing a plurality of reagents on at least a subset of the plurality of regions. As a non-limiting example, the method may include forming at least a portion of a RDT device by selectively depositing PCL on the substrate.

In some examples, the tetrazine-modified protein is attached to the linker in a configured orientation to permit binding of the target analyte. Additionally, the bioorthogonal tethered protein includes a configured length of the tetrazine-modified protein comprising a chain of a plurality of binding domains to the analyte. For instance, a ligand may be repeated a number of times, such as three times, resulting in a chain of binding domains for the target analyte.

A substrate of an RDT device is described herein. In some examples, an RDT device may be manufactured in a scalable manner, resulting in varied size and numbers of assay devices, such as a micro lateral flow assay (μLFA). As used herein, a micro lateral flow assay refers to or includes an assay device that is capable of detecting pico to femtograms of pathogens, yet scales like a LFA. In various examples, the RDT device may be fabricated using a substrate that has been functionalized to attach to a tetrazine tethered protein. More specifically, the substrate of the RDT device may be configured for a particular assay, so as to maximize binding affinity of an analyte. The substrate may be specifically configured with bioorthogonal tethered proteins that have a particular density (e.g., concentration on the substrate surface), a particular length, and a particular orientation for an analyte of interest. As described more thoroughly herein, proteins may be tethered to the substrate using tetrazine (Tet). For more general information on proteins attached to Tet, and techniques to immobilize tetrazine on porous membranes, reference is made to: US Patent Publication 2021/0072238, published on Mar. 11, 2021, and entitled “Immobilization of proteins with controlled orientation and load”, which is herein incorporated by reference in its entirety for its teachings; and to PCT publication WO 2022/109075, published on May 27, 2022, and entitled “Configurable Substrate of a Fluidic Device”, which is attached herein and incorporated by reference in its entirety for its teaching. The preparation of representative tetrazine non-canonical amino acids, methods for genetic encoding proteins and polypeptides using the tetrazine non-canonical amino acids, and proteins and polypeptides comprising the tetrazine non-canonical amino acids is described in PCT publication WO 2016/176689, published on Nov. 2, 2016, entitled “Reagents and methods for bioorthogonal labeling of biomolecules in living cells”, corresponding to US Patent Publication 2019/0077776, published on Mar. 14, 2019, the entire teachings of which are incorporated herein by reference in their entirety.

Generally, manufacturing an RDT device includes a plurality of steps that may be performed in various orders. In some examples, the methods of manufacturing includes creating a region, where reagents are to be dispensed onto the substrate.

A variety of different pathogens (e.g., target analytes) may be detected using example RDTs described herein. Example pathogens include, but are not limited to, viruses and bacteria, such as coronaviruses (e.g., COVID-19), Ebola, dengue, human immunodeficiency virus (HIV), Hantavirus, Lyme disease, Japanese encephalitis, Lassa fever, rabies, Middle Eastern Respiratory Syndrome (MERS), Severe Acute Respiratory Syndrome (SARS), rotavirus, Hepatitis C, yellow fever, Rift Valley fever, Crimean-Congo hemorrhagic fever and other Arenaviruses, Clostridioides difficile, Candida auris, Carbapenem-resistant Acinetobacter, Carbapenem-resistant Enterobacteriaceae, Drug-resistant Neisseria gonorrhoeae, Drug-resistant Camplyobacter, Drug-resistant Candida, ESBL-producing Enterobacteriaceae, Vancomycin-resistant Enterococci (VRE), Drug-resistant nontyphoidal Salmonella, Drug-resistant Salmonella serotype Typhi, Drug-resistant Shigella, Methicillin-resistant Staphylococcus aureus (MRSA), Drug-resistant Streptococcus pneumoniae, Drug-resistant Tuberculosis, Erythomycin-resistant Group A Streptococcus, Clindamycin-resistant Group B Streptococcus, Azole-resistant Aspergillus fumigatus, Drug-resistant Mycoplasma genitalium, Drug-resistant Bordetella pertussis. For more general and specific information on example super bugs, reference is made to https://www.cdc.gov/drugresistance/biggest-threats.html, which is incorporated herein by reference in its entirety.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

### EXPERIMENTAL EMBODIMENTS

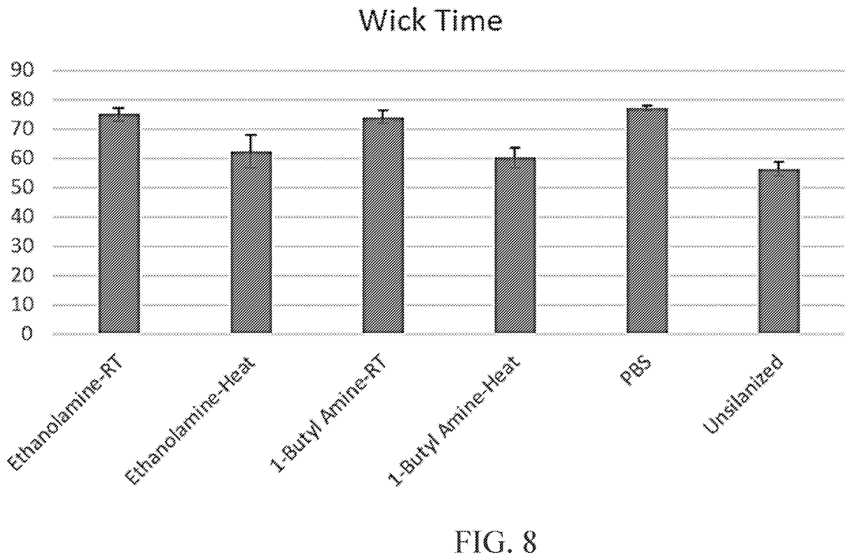

As further illustrated below in connection with the experimental embodiments, a substrate for a RDT was modeled and tested, evidencing the capability to form a RDT device capable of detecting a particular analyte, sensitive enough to detect small volumes of the analyte, and scalable for mass-production and use in a point-of-care setting. FIGS. 8-16 illustrate example results of forming at least portions of an RDT.

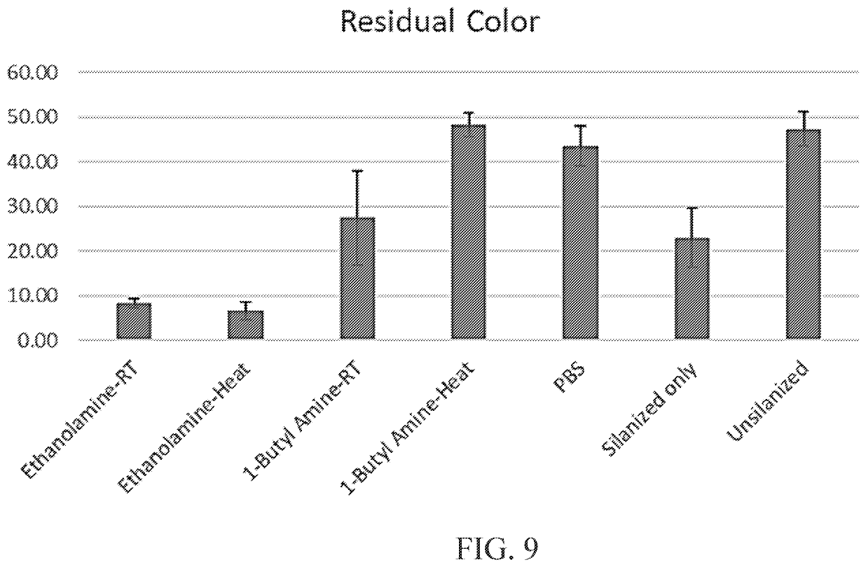

In various experiments, a substrate was modified with a coupling agent to include reactive moieties and then further modified with blocking agents or other reactive compounds to modify flow characteristics of the substrate. Examples of reactive compound used to modify the flow characteristics of a silane functionalized GMF as shown in FIG. 8. As compared with the unsilanized membrane, both ethanolamine and 1-butylamine reacted with epoxide silanized GMF showing increased wick time, e.g., slower wick rate. In addition, higher treatment temperature showed lower wick time than ambient temperature. Thus, the flow rate of a fluid was manipulated through the treatment and treatment conditions. Even though the impact on flow rate is similar between ethanolamine and 1-butylamine treatment, the blocking effect against non-specific binding is not the same. As measured by residual color (FIG. 9) of AuNP/protein complex after its application on treated strips and followed by wicking a fluid, ethanolamine has much lower residual color potentially indicating better blocking ability toward non-specific binding. FIG. 9 further shows an unexpected and somewhat surprising result of the epoxide silanized membrane showing less residual color than the unsilanized one.

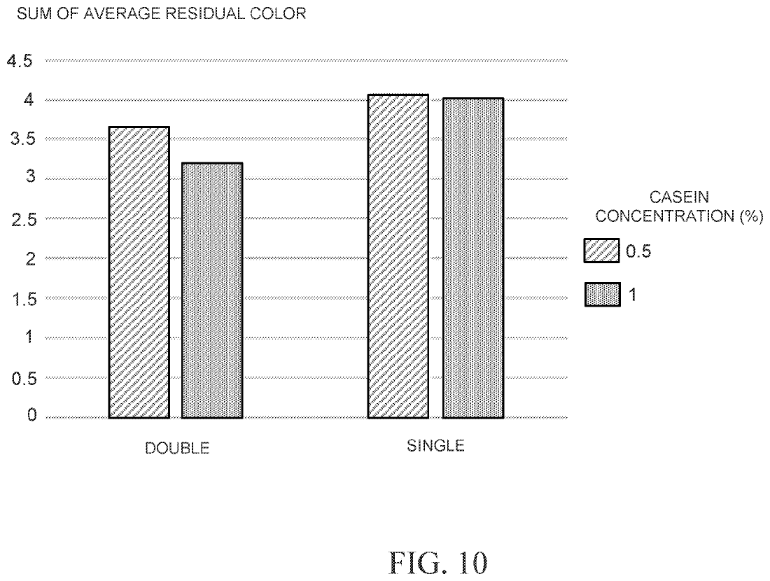

In terms of releasing of AuNP after it is placed on the substrate, somewhat surprisingly, the way the AuNP was deposited affects the residual color as shown in FIG. 10. Double pass printing when AuNP is printed on the substrate released more than that of single pass when casein is used as the blocking agent.

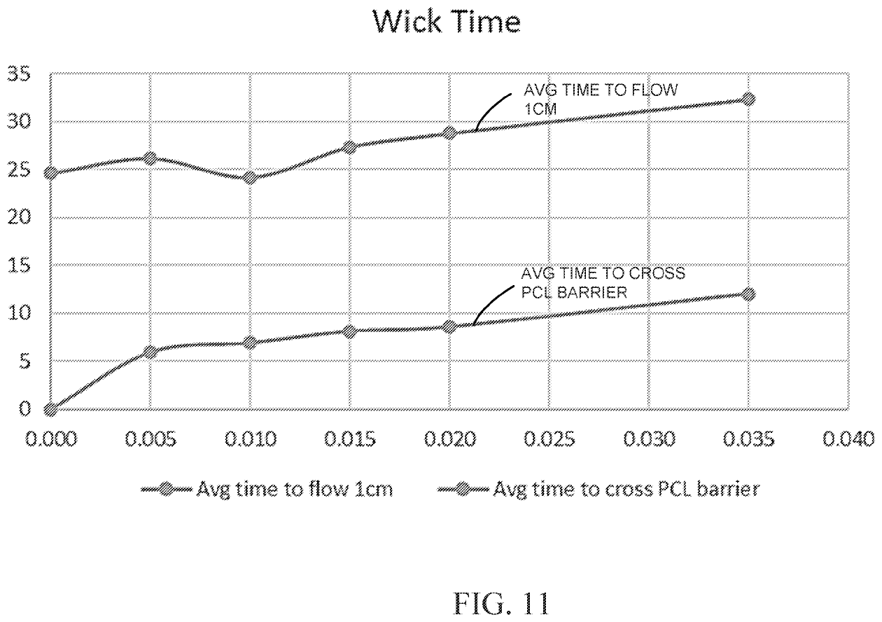

As described above, using the same substrate for different functions in a RDT may present challenges for flow rate. Traditional assays use different materials with different flow rate. Several methods were tested to show the effects of control. In addition to that described above with regards to reactive compounds, barriers using sugar, PEG, and different concentrations of PCL (polycaprolatone) (FIG. 11) have been tested. All show some level of increasing wick time, e.g., reducing flow rate. Reduced flow rate may allow for the additional time for binding with the analyte protein and/or target protein.

As described above, one or more of the detection particles reacted with the target analyte to form a detectable indicator that was used to detect the presence of the analyte in the sample. The detectable indicator may include an optical indicator (e.g., visual, CCD detectable, detection using smartphone and enhanced with digital manipulation application, fluorescence, chemiluminescent), an electrical indicator (e.g., Amperometric, potentiometric, impedance), a radiometric indicator (e.g., Geiger counter, x-ray file, etc.), and/or a Plasmon Resonance (SERS).

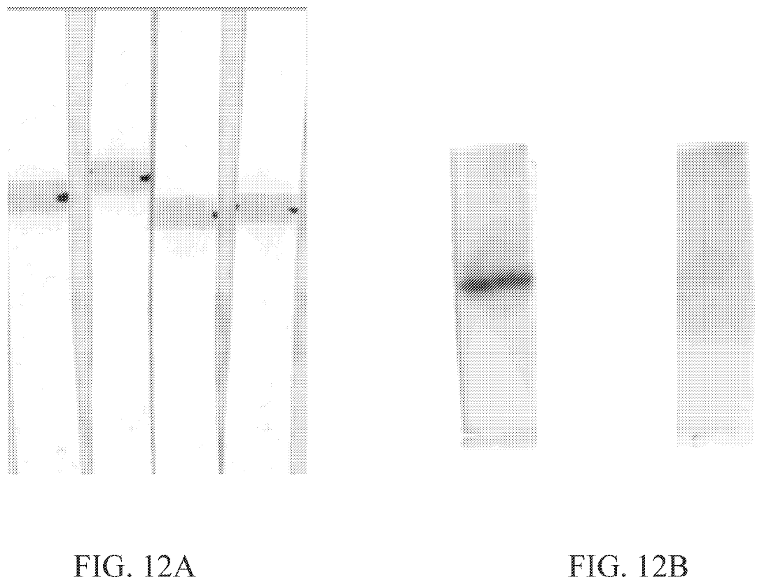

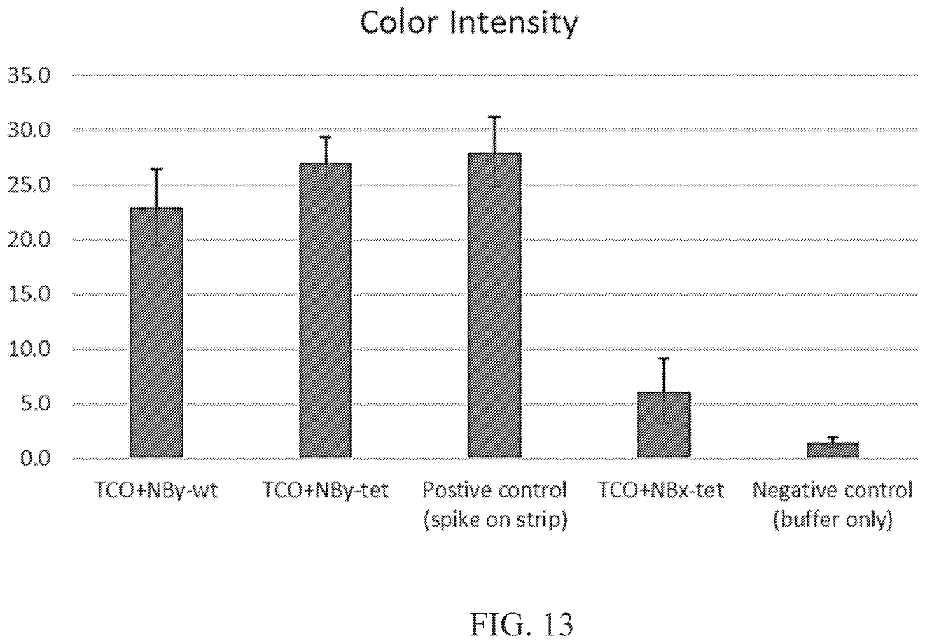

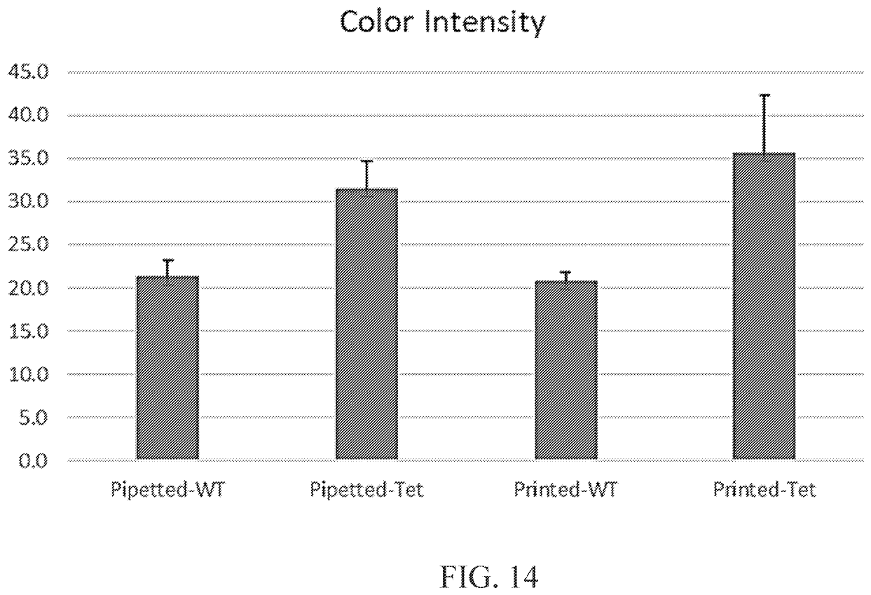

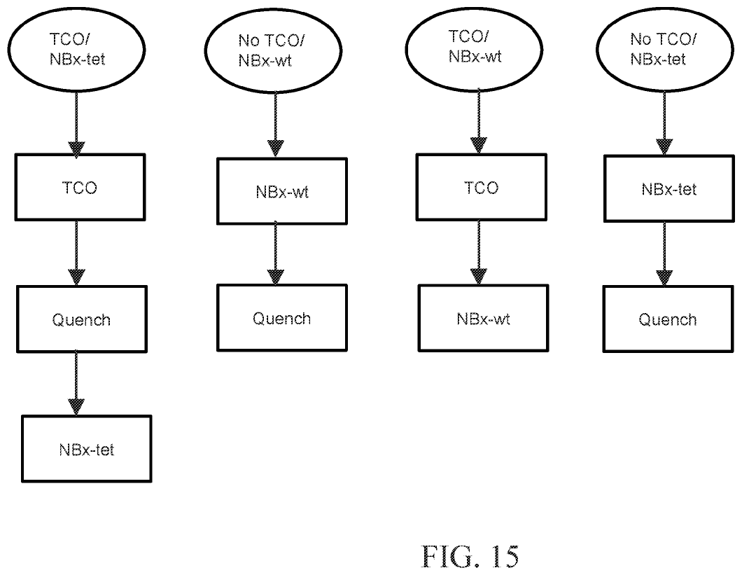

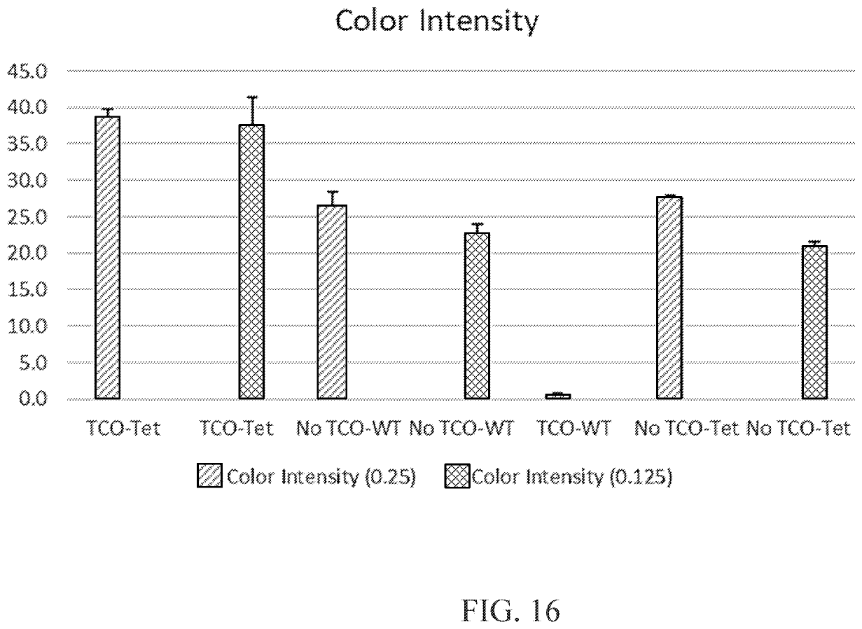

In some experimental examples, upon applying an analyte, IgG, as shown in FIG. 12A, followed by applying a detection particle, AuNP, the test region showed the color of AuNP, indicating a successful run. The test region may be analyzed using colorimetric values to quantify the results in the form of color density. Subsequently, a test using another analyte, spike protein, was successfully completed and analyzed (FIG. 13). FIG. 13 also demonstrates the benefit of using tetrazine-modified protein over a protein without the tetrazine in the test region in the form of higher signals strength. FIG. 14 shows deposition of the capture protein by printing yielded similar or stronger signal than that by pipetting in addition to stronger signal using tet-protein than protein without the tetrazine. FIG. 15 is an example of controlled deposition of label protein with tetrazine on AuNP over that without tetrazine. Again, stronger signal was obtained than that without tetrazine (FIG. 16).

In further examples, as shown by FIG. 12B, a tet-protein (e.g., tet-protein A3) was printed on the test line, and upon applying an analyte, IgG, followed by apply a detection particle comprising protein A B1 linked to a fluorescent dye via tet, The test region showed the color of the fluorescent dye, as illustrated by the left side of FIG. 12B. The right side of FIG. 12B illustrates a negative control in which no tet protein (tet-protein A3) was printed on the test line as the capture agent, and which showed no fluorescent dye signal.

In various examples, a digital dispensing device was used for reagent printing of at least some of the reagents and was used in validating and manufacturing the assay device. Solutions for a protein loading and partial loading protocol consistent with the present disclosure are included below in Tables 1 and 2.

TABLE 1
Reagents for Protein Loading Protocol

| | Viscosity at | Thermal |
|---|---|---|
| Reagent | 25° C. (mPa-s) | Stability |
| Water | 0.89 | b.p. 100° C. |
| EtOH | 0.98 | b.p. 78.37° C. |
| 2M NaOH | >0.89 | b.p. 100° C. |
| Trimethoxysilane | ~0.56 | b.p. 110.6° C. |
| TCO amine | ~0.98 | Stable at 4° C. |
| Tween Buffer | >0.89 | c.p. 76° C. |
| Proteins or Protein | >0.89 | ~60° C. |
| Fragments | | |
| Blocking Agent(s) | various | various |
| Sucrose or Glucose | >0.89 | <100° C. |

TABLE 2
Reagents for Detection Particle Label Protein Loading

| | Viscosity at | Thermal |
|---|---|---|
| Reagent | 25° C. (mPa · s) | Stability |
| TCO amine | ~0.98 | Stable at 4° C. |
| PO4 | 0.98 | b.p. 78.37° C. |
| NaCL | >0.89 | b.p. 100° C. |
| Protein or Protein | >0.89 | ~60° C. |
| Fragments | | |
| Blocking Agent(s) | unknown | unknown |
| (e.g., Quench | | |
| Solution) | | |
| Buffer | >0.89 | c.p. 76° C. |
| Sucrose or Glucose | >0.89 | <100° C. |

Note that the following are abbreviations: b.p is for boiling point, and c.p. is for cloud point.

In some examples, the reagents were loaded on the RDT device by treating the surface of the substrate with NaOH, followed by trimethoxysilane and which results in trimethoxysilane bound on the surface. The surface was then treated with TCO-NH2, resulting in TCO being bound to the silane. The substrate surface was then treated with the tetrazine-modified protein and the tet-PEG polymer (e.g., Tet-PEG-5K), resulting in the bioorthogonal tethered protein being tethered to a first portion of the volume of TCO and the tet-PEG polymer being bound to a second portion of the volume of TCO. The surface was then treated with another blocking agent. In some examples, the tet-PEG was not used. In various examples, casein was used as a blocking agent which was applied at least in the sample input region after the tet-protein was deposited and before the detection particles are deposited.

Genetic code expansion (GCE), is of great use in biomedical research and development of therapeutics. Bioconjugate materials that incorporate site-specifically incorporated ncAAs form well-organized, highly uniform biomolecular monolayers spontaneously. Compared to conventional immobilization strategies, substrates of the present disclosure increase protein binding efficiency, signal fidelity and reliability because: 1. Bioorthogonal orientation and length. 2. Controlling protein surface concentration, e.g., controlled loading, and uniformity closely approximates pseudo first-order binding kinetics.

1. A rapid detection test (RDT) apparatus, comprising:
a substrate at least partially coated with a coupling agent having functional groups;
a test region disposed on a first portion of the substrate, the test region including a set of capture agents configured to bind to a target analyte in a biological sample;
a control region disposed on a second portion of the substrate, the control region including a set of control agents, each of the control agents including an analyte protein; and
a set of detection particles that exhibit a detectable label, each of the detection particles configured to bind to at least one of the target analyte or the analyte protein of the set of control agents, wherein the RDT apparatus further includes at least one of:
each of the capture agents include first bioorthogonal tethered proteins including:
a first tetrazine-modified protein, wherein the first tetrazine-modified protein includes a first ligand configured to bind to the target analyte; and
a first linker bound to the first tetrazine-modified protein and functional groups of the coupling agent disposed in the test region; or
each of the detection particles including a second tetrazine-modified protein which includes a second ligand configured to bind to at least one of the target analyte or the analyte protein of the set of control agents.

2. The RDT apparatus of claim 1, wherein the RDT apparatus includes each of:
the set of capture agents each include the first bioorthogonal tethered protein, and
the detection particles each include the second tetrazine-modified protein.

3. The apparatus of claim 1, wherein each of the detection particles include a particle comprising:
a bead, a nanoparticle, or a dye.

4. The apparatus of claim 1, wherein the set of detection particles are disposed on a sample input region configured to receive the biological sample, wherein the test region and the control region are downstream from the sample input region of the substrate.

5. The apparatus of claim 1, further including a sample container that includes a solution with the set of detection particles, the sample container configured to receive the biological sample and to provide the biological sample and the set of detection particles to a sample input region of the substrate.

6. The apparatus of claim 1, wherein each of the control agents further include a second linker bound to the analyte protein and to the second portion of the substrate.

7. (canceled)

8. The apparatus of claim 1, wherein the substrate is formed of glass microfibers (GMF).

9. The apparatus of claim 1, wherein the first linker is selected from:
trans-cyclooctene (TCO), a TCO derivative, a TCO with an amine moiety, a TCO with a carboxylic acid moiety, a norbornene anhydride, a norbornene with an amine moiety, and a norbornene with a carboxylic acid moiety.

10. The apparatus of claim 1, wherein each of the set of control agents include a second bioorthogonal tethered protein and the analyte protein includes a third tetrazine-modified protein.

11. The apparatus of claim 10, wherein each detection particles of the set of detection particles further include a second linker bound to the second tetrazine-modified protein and the particle.

12-13. (canceled)

14. The apparatus of claim 19, wherein each detection particle of the set of detection particles is attached to the second tetrazine-modified protein having the same type of second ligand.

15. The apparatus of claim 1, further including a flow control agent disposed on at least one of the substrate, the detection particles, or portions thereof.

16. A rapid detection test (RDT) device, comprising:
a substrate at least partially coated with a coupling agent having functional groups;
a test region disposed on a first portion of the substrate, the test region including a set of capture agents configured to bind to a target analyte in a biological sample;
a control region disposed on a second portion of the substrate, the control region including a set of control agents, each of the control agents including an analyte protein; and
a sample input region disposed on a third portion of the substrate, the sample input region including a set of detection particles that exhibit a detectable label and are configured to bind to at least one of the target analyte or the analyte protein of the set of control agents, wherein the RDT device further including at least one of:
each of the set of capture agents include a first bioorthogonal tethered protein including:
a first tetrazine-modified protein, wherein the first tetrazine-modified protein includes a first ligand configured to bind to the target analyte; a first linker bound to the first tetrazine-modified protein and functional groups of the coupling agent disposed in the test region; or
each of the detection particles including a second tetrazine-modified protein which includes a second ligand configured to bind to at least one of the target analyte or the analyte protein of the set of control agents.

17. (canceled)

18. The RDT device of claim 16, wherein each of the detection particles include a particle comprising:
a bead, a nanoparticle, or a dye.

19. The RDT device of claim 16, wherein the set of detection particles are disposed on a sample input region configured to receive the biological sample, wherein the test region and the control region are downstream from the sample input region of the substrate.

20-21. (canceled)

22. The RDT device of claim 16, wherein the substrate is formed of glass microfibers (GMF), a polymer, or a metal.

23. (canceled)

24. The RDT device of claim 16, wherein each of the set of control agents include a second bioorthogonal tethered protein and the analyte protein includes a third tetrazine-modified protein.

25-28. (canceled)

29. The RDT device of claim 16, further including a flow control agent disposed on at least one of the substrate, the detection particles, or portions thereof.

30-51. (canceled)

52. A rapid detection test (RDT) apparatus, comprising:
a glass microfibers (GMF) substrate at least partially coated with a silane coupling agent having functional groups;
a test region disposed on a first portion of the substrate, the test region including a set of capture agents configured to bind to a target analyte in a biological sample, each of the capture agents including a first ligand configured to bind to the target analyte; and
a control region disposed on a second portion of the substrate, the control region including a set of control agents, each of the control agents including an analyte protein; and
a set of detection particles that exhibit a detectable label, each of the detection particles including a label protein, wherein the label protein includes a second ligand configured to bind to at least one of the target analyte or the analyte protein of the set of control agents.

53-60. (canceled)

61. The RDT apparatus of claim 52, wherein at least one of:
each of the set of capture agents include first bioorthogonal tethered proteins including:
a first tetrazine-modified protein, wherein the first tetrazine-modified protein includes a first ligand configured to bind to the target analyte; and
a first linker bound to the first tetrazine-modified protein and functional groups of the coupling agent disposed in the test region; or
each of the label proteins including a second tetrazine-modified protein which includes a second ligand configured to bind to at least one of the target analyte or the analyte protein of the set of control agent.

62-105. (canceled)
